(19) United States
(12) Patent Application Publication
(10) Pub. No.: US 2012/0093726 A1
(43) Pub. Date: Apr. 19, 2012

(54) Radiolabeled Fluorine Derivatives of Methionine

(76) Inventors: Andrew Katsifis, Sydney (AU); Christopher John Reginald Fookes, New South Wales (AU); Ivan Greguric, New South Wales (AU); Thomas Bourdier, New South Wales (AU)

(21) Appl. No.: 13/259,549

(22) PCT Filed: Mar. 24, 2009
(86) PCT No.: PCT/AU09/00333

§ 371 Date: Dec. 22, 2011

(51) Int. Cl. A61K 51/04; C07C 319/20; C07C 323/58

(52) U.S. Cl. 424/1.89; 562/557; 560/153

(57) ABSTRACT

The invention provides compound which is an ¹⁸F-radiolabelled S-propylhomocysteine or a derivative thereof. The compound has an enantiomeric purity of at least about 90%. ¹⁸F-radiolabelled S-propylhomocysteine may be made by treating an N-protected ester of a substituted S-propylhomocysteine with a complexed F″ salt in the presence of a base to form a protected product and then deprotecting the protected product to form the ¹⁸F-radiolabelled S-propylhomocysteine. In this method the N-protected ester has a leaving group on the S-propyl group and has an enantiomeric purity of at least about 90%. The base should be such that it does not cause racemisation of the protected product.

### TECHNICAL FIELD

The present invention relates to radiolabelled fluorine compounds and to methods for making them.

### BACKGROUND OF THE INVENTION

[¹⁸F]Fluoro-2-deoxy-D-glucose (FDG) is the most widely used radiotracer in tumour diagnosis using Positron Emission Tomography (PET). However, clinical studies have demonstrated that in some instances, it is difficult to differentiate neoplasms from inflammatory tissues. Radiolabelled amino acids, particularly those incorporating the PET radionuclides carbon-11 and fluorine-18 have been used to overcome some of the disadvantages of FDG.

For this purpose, radiolabelled amino acids and in particular S-(2-[¹¹C]methyl)-L-methionine ([¹¹C]MET), have been extensively used in PET tumour imaging in both animals and man, however the short half life of carbon-11 (t_{1/2}=20 min) limits the widespread application of these tracers. This has prompted the development of ¹⁸F-labelled amino acids (t_{1/2}=110 min) such as 4-[¹⁸F]fluoro-L-phenylalanine and 2-[¹⁸F]fluoro-L-tyrosine. These amino acids demonstrated suitable imaging characteristics, but the radiosynthesis of these compounds is difficult, long and the radiochemical yield is low.

O-(2-[¹⁸F]fluoroethyl)-L-tyrosine ([¹⁸F]FET), has demonstrated good imaging properties in differentiating neoplasms and inflammatory tissue in the brain and has become a lead candidate in the development of ¹⁸F-labelled amino acids. However, its application in imaging neoplasms in the periphery of the body has not been very successful, primarily from a lack of specificity and poor target to non-target ratios. Hence the development of other amino acids that display high tumour uptake and low background in the surrounding tissue would be of clinical value. Recently, other amino acids radiolabelled with both Carbon-11 and Fluorine-18 have been published and their biological properties in vivo investigated, including the new fluorinated derivative of the amino acid methione (MET) S-(2-[¹⁸F]fluoroethyl)-L-homocysteine ([¹⁸F]FEHCys). However, [¹⁸F]FEHCys was prepared via a two step synthesis and displayed considerable instability in aqueous systems, rendering its potential use in clinical medicine unlikely.

There is therefore a need for a new analogue of [¹⁸F]FEHCys with improved stability in aqueous systems and a process for preparing the analogue in a routine clinical or radiopharmaceutical environment.

### OBJECT OF THE INVENTION

It is the object of the present invention to substantially overcome or at least ameliorate one or more of the above disadvantages.

### SUMMARY OF THE INVENTION

In a first aspect of the invention there is provided a compound which is an ¹⁸F-radiolabelled S-propylhomocysteine or a derivative thereof. The compound may have an enantiomeric purity of at least about 90%.

The following options may be used in conjunction with the first aspect, either individually or in any suitable combination.

The compound may be ¹⁸F-radiolabelled on the S-propyl group. It may be S-(3-[¹⁸F]fluoropropyl)homocysteine. It may be an N-protected S-(3-[¹⁸F]fluoropropyl)homocysteine, e.g. protected as an N-t-butyloxycarbonyl compound. It may be C-protected as an ester, e.g. a t-butyl ester. It may be both N-protected and C-protected. It may for example be N-t-butoxycarbonyl-S-(3-[¹⁸F]fluoropropyl)homocysteine t-butyl ester.

The compound may be a D-enantiomer (i.e. at least about 90% D-enantiomer). It may be S-(3-[¹⁸F]fluoropropyl)-D-homocysteine. It may be N-t-butoxycarbonyl-S-(3-[¹⁸F]fluoropropyl)-D-homocysteine t-butyl ester.

The compound may have a radiochemical purity of at least about 90%.

In a second aspect of the invention there is provided a process for making an ¹⁸F-radiolabelled S-propylhomocysteine comprising:
treating an N-protected ester of a substituted S-propylhomocysteine having a leaving group on the S-propyl group with a complexed ¹⁸F⁻ salt in the presence of a base to form a protected product, said ester having an enantiomeric purity of at least about 90% and said base being such that it does not cause racemisation of the protected product; and
deprotecting the protected product to form the ¹⁸F-radiolabelled S-propylhomocysteine.

The following options may be used in conjunction with the second aspect, either individually or in any suitable combination.

The N-protected ester may be an N-t-butoxycarbonyl (Boc) protected ester. It may be a t-butyl ester.

The leaving group may be in the 3 position of the S-propyl group.

The N-protected ester of the substituted propyl homocysteine may be that enantiomer which is capable of producing the D-enantiomer of the ¹⁸F-radiolabelled S-propylhomocysteine under the conditions of said process.

The leaving group may be chloride, bromide, mesylate or tosylate.

The base may be a weak base. It may be a weaker base than carbonate. It may be a sufficiently weak base that it does not cause abstraction of the α-hydrogen atom or the N-protected ester of the substituted S-propylhomocysteine under the conditions of the reaction. The base may be tetra-n-butylammonium hydrogen carbonate. The base may an oxalate salt. It may be potassium oxalate. The process may be conducted in the absence of be a base which is capable of racemising the N-protected ester of the substituted S-propylhomocysteine or the protected product to a substantial degree under the conditions used in the process. It may be conducted in the absence of carbonate.

The step of deprotecting may comprise treating the protected product with a strong acid, e.g. hydrochloric acid.

The complexed ¹⁸F⁻ salt may be a cryptand-complexed ¹⁸F salt. It may be a 4,7,13,16,21,24-hexaoxa-1,10-diazabicyclo[8.8.8]-hexacosane-complexed ¹⁸F⁻ salt. It may be a salt derived from a tetra-n-butylammonium salt, such as tetra-n-butylammonium hydrogencarbonate, and [18F] fluoride. The tetra-n-butylammonium salt may be incapable of racemising the N-protected ester of the substituted S-propylhomocysteine. It may be a sufficiently weak base that it does not cause abstraction of the α-hydrogen atom or the N-protected ester of the substituted S-propylhomocysteine under the conditions of the reaction. The complexed ¹⁸F salt may be tetra-n-butyl ammonium [18F]fluoride. It may be formed in situ or it may be preformed.

The step of deprotecting may be conducted without separation of the protected product.

The process may comprise the step of making the N-protected ester of the substituted S-propylhomocysteine having a leaving group on the propyl group. This step may comprise reacting an N-protected homocysteine ester with a substituted 1-bromopropane. In this context it will be understood that the 1-bromopropane has a substituent that is not the 1-bromosubstituent.

In some embodiments, either:

the substituent on the 1-bromopropane is the leaving group, or

the substituent on the 1-bromopropane is an OH group, in which case the step of making the N-protected ester of the substituted propyl homocysteine having a leaving group on the propyl group may additionally comprise tosylating the OH group (i.e. converting the OH group to a p-toluenesulfonate group or a methanesulfonate group.

The process may comprise making the N-protected homocysteine ester from homocystine.

The process may additionally comprise purifying the ¹⁸F-radiolabelled S-propylhomocysteine.

The process may produce the ¹⁸F-radiolabelled S-propylhomocysteine in an enantiomeric purity of at least about 90%. It may produce the ¹⁸F-radiolabelled S-propylhomocysteine in a radiochemical purity of at least about 90%.

In an embodiment there is provided a process for making S-(3-[¹⁸F]fluoropropyl)-D-homocysteine comprising:
treating N-t-butoxycarbonyl-S-(3-tosyloxypropyl)-D-homocysteine t-butyl ester with a cryptand-complexed ¹⁸F salt in the presence of oxalate to form a protected product; and
deprotecting the protected product with hydrochloric acid to form the S-(3-[¹⁸F]fluoropropyl)-D-homocysteine.

In another embodiment there is provided a process for making S-(3-[¹⁸F]fluoropropyl)-D-homocysteine comprising:
reacting N-t-butoxycarbonyl-D-homocysteine t-butyl ester with 3-bromo-1-propanol to form N-t-butoxycarbonyl-S-(3-hydroxypropyl)homocysteine t-butyl ester;
tosylating the N-t-butoxycarbonyl-S-(3-hydroxypropyl)-D-homocysteine t-butyl ester to form N-t-butoxycarbonyl-S-(3-tosyloxypropyl)-D-homocysteine t-butyl ester;
treating the N-t-butoxycarbonyl-S-(3-tosyloxypropyl)-D-homocysteine t-butyl ester with a cryptand-complexed ¹⁸F salt in the presence of oxalate to form a protected product; and
deprotecting the protected product with hydrochloric acid to form the S-(3-[¹⁸F]fluoropropyl)-D-homocysteine.

In a third aspect of the invention there is provided use of the compound of the first aspect, or made by the second aspect, as a radiotracer in tumour diagnosis.

In a fourth aspect of the invention there is provided use of the compound of the first aspect, or made by the second aspect, in the manufacture of a composition for use in positron emission tomography.

In a fifth aspect of the invention there is provided a composition comprising the compound of the first aspect, or made by the second aspect, and a clinically acceptable carrier. The carrier may be an aqueous carrier. It may be a buffered aqueous carrier.

### BRIEF DESCRIPTION OF THE DRAWINGS

A preferred embodiment of the present invention will now be described, by way of an example only, with reference to the accompanying drawings wherein:

FIG. 1 is a scheme for synthesis of precursors 1-3—reagents and conditions: (a) Boc₂O, dioxane, Na₂CO_{3(aq)}, RT; (b) tort-butyl-2,2,2-trichloroacetimidate, CH₂Cl₂, RT; (c) tributylphosphine, DMF, RT; (d) 1-bromo-3-halopropane or 3-bromopropanol, K₂CO₃, DMF, RT; (e) p-toluenesulfonyl chloride, N,N,N′,N′-tetramethyl-1,6-hexanediamine, acetonitrile, RT;

FIG. 2 is a scheme for radiosynthesis of [¹⁸F]FPHCys;

FIG. 3 is a graph showing radiolabelling of tosyl, bromo and chloro-precursors 1-3 (5 mg) in acetonitrile (1 mL) with K₂₂₂ (10 mg) and K₂CO₃ (2 mg) as a function of temperature and time;

FIG. 4 is a graph showing radiolabelling of tosyl, bromo and chloro-precursors 1-3 (5 mg) in acetonitrile (1 mL) with K₂₂₂ (10 mg), K₂C₂O₄ (2.55 mg) and K₂CO₃ (50 μg) as a function of temperature and time;

FIG. 5 shows a typical chromatogram of [¹⁸F]FPHCys using both gamma and UV detection;

FIG. 6 shows a chromatogram (gamma detection) on a chiral column of [¹⁸F]-L-FPHCys made using carbonate as base

FIG. 7 shows a chromatogram (gamma detection) on a chiral column of [¹⁸F]-L-FPHCys made using oxalate as base;

FIG. 8 shows a chromatogram (UV detection) on a chiral column of [¹⁸F]-L-FPHCy made using oxalate as base;

FIG. 9 shows a chromatogram (gamma detection) on a chiral column of [¹⁸F]-D-FPHCys made using oxalate as base;

FIG. 10 shows a chromatogram (UV detection) on a chiral column of [¹⁸F]-D-FPHCy made using oxalate as base;

FIG. 11 shows a chromatogram (gamma detection) on a chiral column of [¹⁸F]-D-FPHCys at t=0 in ethanol/buffer;

FIG. 12 shows a chromatogram (UV detection) on a chiral column of [¹⁸F]-D-FPHCy at t=0 in ethanol/buffer;

FIG. 13 shows a chromatogram (gamma detection) on a chiral column of [¹⁸F]-D-FPHCys at t=4 h in ethanol/buffer;

FIG. 14 shows a chromatogram (UV detection) on a chiral column of [¹⁸F]-D-FPHCy at t=4 h in ethanol/buffer;

FIG. 15 shows a chromatogram (gamma detection) on a chiral column of [¹⁸F]-D-FPHCys at t=0 in saline;

FIG. 16 shows a chromatogram (UV detection) on a chiral column of [¹⁸F]-D-FPHCy at t=0 in saline;

FIG. 17 shows a chromatogram (gamma detection) on a chiral column of [¹⁸F]-D-FPHCys at t=4 h in saline;

FIG. 18 shows a chromatogram (UV detection) on a chiral column of [¹⁸F]-D-FPHCy at t=4 h in saline;

FIG. 19 shows a chromatogram (gamma detection) on a chiral column of [¹⁸F]-D-FPHCys at t=0 in Dulbecco's phosphate buffered saline;

FIG. 20 shows a chromatogram (UV detection) on a chiral column of [¹⁸F]-D-FPHCy at t=0 in Dulbecco's phosphate buffered saline;

FIG. 21 shows a chromatogram (gamma detection) on a chiral column of [¹⁸F]-D-FPHCys at t=4 h in Dulbecco's phosphate buffered saline;

FIG. 22 shows a chromatogram (UV detection) on a chiral column of [¹⁸F]-D-FPHCy at t=4 h in Dulbecco's phosphate buffered saline;

FIG. 23 shows a chromatogram (gamma detection) on a chiral column of [¹⁸F]-D-FPHCys at t=0 in PBS;

FIG. 24 shows a chromatogram (UV detection) on a chiral column of [¹⁸F]-D-FPHCy at t=0 in PBS;

FIG. 25 shows a chromatogram (gamma detection) on a chiral column of [¹⁸F]-D-FPHCys at t=4 h in PBS;

FIG. 26 shows a chromatogram (UV detection) on a chiral column of [¹⁸F]D-FPHCy at t=4 h in PBS;

FIG. 27 shows uptake of¹⁸F-fluoro-L-propylmethionine by various types of tumour cells

FIG. 28 shows uptake of ¹⁸F-fluoro D-propylmethionine by various types of tumour cells

FIG. 29 shows inhibition of ¹⁸F-fluoro-L-propylmethionine transport by known inhibitors;

FIG. 30 shows inhibition of ¹⁸F-fluoro-D-propylmethionine transport by known inhibitors;

FIG. 31 shows D-[¹⁸F]FPM uptake in A431 tumoured mice: the tracer displayed a percent injected dose (% ID) of 1.96% with a tumour to background ratio of 7.86;

FIG. 32 shows D-[¹⁸F]FPM uptake in HT29 tumoured mice: the tracer displayed a percent injected dose (% ID) of 0.75% with a tumour to background ratio of 2.10;

FIG. 33 shows L-[¹⁸F]FPM uptake in A431 tumoured mice: the tracer displayed a percent injected dose (% ID) of 2.12% with a tumour to background ratio of 5.39;

FIG. 34 shows L-[¹⁸F]FPM uptake in HT29 tumoured mice: the tracer displayed a percent injected dose (% ID) of 2.13% with a tumour to background ratio of 2.45;

FIG. 35 FDG uptake in A431 tumoured mice: the tracer displayed a percent injected dose (% ID) of 5.84% inthis tumour with a tumour to background ratio of 3.33;

FIG. 36 shows uptake of FDG uptake in HT29 tumoured mice: the tracer displayed a percent injected dose (% ID) of 2.37% with a tumour to background ratio of 2.11; and

FIG. 37 shows a tumour autoradiographic study of L-FPM (above) and photographic image (bellow).

### DETAILED DESCRIPTION OF PREFERRED EMBODIMENTS

The invention relates to ¹⁸F-radiolabelled S-propylhomocysteine and derivatives thereof. In the present specification the stereochemistry of various compounds is denoted using D/L terminology. The prefix S-, used in conjunction with propyl or substituted propyl groups, is used to denote that the specified group is attached to a sulfur atom, and is unrelated to stereochemistry.

The compounds of the invention may have an enantiomeric purity of at least about 90%, or at least about 91, 92, 93, 94, 95, 96, 97, 98 or 99%. They may be useful as tumour imaging agents, however certain precursors to the compounds are also encompassed by the invention. In particular, compounds having protecting groups on the N(NH₂) or C(COOH) of the homocysteine are also encompassed. Thus for example a compound according to the invention may be an N-t-butoxycarbonyl derivative, or a t-butyl ester, or both an N-t-butoxycarbonyl derivative and a t-butyl ester. Other embodiments of the invention include the unprotected ¹⁸F-radiolabelled S-propylhomocysteine, in which the NH₂ group and the COOH group have no substituents. It will be understood that, depending on the pH of the surrounding medium, one or both of the NH₂ group and the COOH group may be ionised, i.e. the NH₂ group may be present as NH₃⁺ and the COOH group may be present as COO⁻. The compound may therefore be present as an ammonium salt, or a carboxylate salt, or as a zwitterion.

The compound may be ¹⁸F-radiolabelled on the S-propyl group. It may have a single ¹⁸F per molecule, in particular per S-propyl group. The ¹⁸F may be a terminal ¹⁸F. It may be in the 3-position of the S-propyl group.

It may be a D-enantiomer. It may be at least about 90% D-enantiomer, or at least about 91, 92, 93, 94, 95, 96, 97, 98 or 99%.

The compound may have a radiochemical purity of at least about 90%, or at least about 91, 92, 93, 94, 95, 96, 97, 98 or 99%. It may have both radiochemical and enantiomeric purity of at least about 90%, or at least about 91, 92, 93, 94, 95, 96, 97, 98 or 99%.

The ¹⁸F-radiolabelled S-propylhomocysteine may be made by treating an N-protected ester of a substituted S-propylhomocysteine having a leaving group on the S-propyl group with a complexed ¹⁸F⁻ salt in the presence of a base to form a protected product. The protected product may then be deprotected to form the ¹⁸F-radiolabelled propylhomocysteine. These steps may be conducted in a “one pot” process, i.e. without isolation or purification of the protected product.

The ester group may represent a carboxyl group having a protecting group attached thereto. The N-protected ester may be an N-t-butoxycarbonyl (Boc) protected ester. It may be a t-butyl ester.

The leaving group may be in the 3 position of the S-propyl group. It may be in some other position, e.g. the 1 position or the 2 position. There will commonly be only a single leaving group in the S-propyl group.

The N-protected ester of the substituted S-propylhomocysteine may be that enantiomer which is capable of producing the D-enantiomer under the conditions of the process.

The leaving group may be a halogen. It may be for example chloride or bromide or iodide. It may be tosylate. It may be mesylate (methanesulfonate). It may be triflate (trifluoromethanesulfonate). It may be azide. It may be thiocyanate.

The base may be an oxalate salt. It may be a potassium salt. It may be a sodium salt. It may be a mild base. It may be potassium oxalate. The base may be a base which does not racemise the N-protected ester of the substituted propyl homocysteine or the protected product. It will be understood that a small amount of racemisation may be tolerated within the scope of the phrase “does not racemise”, and this phrase should be to indicate that the protected product does not racemise to a substantial degree under the conditions used in the process. The base may not racemise the N-protected ester to a degree greater than about 5%, or 4, 3, 2 or 1%. The inventors have surprisingly found that use of an oxalate salt as the base results in a very low level of racemisation. It therefore can lead to a product having a high degree of enantiomeric purity. The D-enantiomer appears to be metabolised in the body of a patient outside a tumour to a lesser degree than the L-enantiomer. Consequently it is considered to be more efficient at reaching a tumour and is therefore considered to be a more efficient labelling processes.

The step of deprotecting may comprise treating the protected product with a strong acid. The acid may be a mineral acid. It may be a strong organic acid. It may for example be trifluoroacetic acid, hydrochloric acid, sulphuric acid, hydrobromic acid or some other suitable acid at a concentration that it will efficiently remove the protecting groups in a timely manner without decomposition of the product. It may have a concentration of at least about 5N, or about 5 to 10 or 5 to 7N, e.g. about 5, 6, 7, 8, 9 or 10N.

The complexed ¹⁸F⁻ salt may be a cryptand-complexed ¹⁸F⁻ salt. It may be a 4,7,13,16,21,24-hexaoxa-1,10-diazabicyclo[8.8.8]-hexacosane-complexed ¹⁸F⁻ salt. It may be a ¹⁸F-salt of tetra-n-butyl ammonium hydrogen carbonate. The ¹⁸F may be about 500 to about 1000 mCi, or about 500 to 800, 700 to 1000, 500 to 600, 900 to 1000 or 600 to 800 mCi, e.g. about 500, 600, 700, 800, 900 or 1000. It may be in some cases less than 500 mCi or greater than 1000 mCi. It may be generated using a Cyclotron or other suitable source.

The step of deprotecting may be conducted without separation of the protected product, i.e. the introduction of the ¹⁸F and the deprotection may be conducted as a one pot process.

The radiochemical yield of the synthesis may be at least about 10%, or at least about 15, 20, 25 or 30%, or may be about 10 to about 100%, or about 10 to 50, 10 to 30, 30 to 50, 50 to 100 or 25 to 35%, e.g. about 10, 15, 20, 25, 30, 35, 40, 45, 50, 60, 70, 80, 90 or 100%.

In an embodiment of the process, activated complexed ¹⁸F is prepared initially. This may be conducted using an activated adsorbent device (e.g. activated with oxalate). Thus The ¹⁸F⁻ may be adsorbed onto the activated adsorbent device, and then desorbed using oxalate or carbonate or a mixture of the two, together with a complexing agent (e.g. the cryptand). The resulting solution contains the desired activated complexed ¹⁸F⁻, and may be dried (e.g. in a stream of dry gas or at high temperature or both, either simultaneously or sequentially). Suitable gases include nitrogen, helium, argon etc. The drying may be conducted under a partial vacuum. The resulting dried activated complexed ¹⁸F⁻ may be used to treat the N-protected ester of the substituted propyl homocysteine, commonly in a solvent. The ester may be present in solvent at about 0.1 to about 1% w/v, or about 0.1 to 0.5, 0.1 to 0.3, 0.2 to 1, 0.5 to 1 or 0.2 to 0.5%, e.g. about 0.1, 0.2, 0.3, 0.4, 0.5, 0.6, 0.7, 0.8, 0.9 or 1%. The solvent may be a polar solvent. It may be aprotic. It may be for example acetonitrile, diethyl ether, ethylene carbonate, tetrahydrofuran, dioxane, dimethylformamide, dimethyl sulfoxide, dimethyl acetamide, diethyl acetamide or some other suitable solvent such as the hindered alcohol tert-butanol or neopentyl alcohol. The reaction may be conducted in a sealed container. It may be conducted under elevated temperature in the sealed vial so that the reaction is under elevated (i.e. above atmospheric) pressure. The reaction may be conducted at a temperature of about 50 to about 150° C., or about 50 to 100, 100 to 150, 80 to 120, 80 to 100, 100 to 120 or 90 to 110° C., e.g. about 50, 60, 70, 80, 90, 100, 110, 120, 130, 140 or 150° C. The reaction may be conducted for a suitable time to achieve the desired conversion. The time may depend on the temperature. It may for example be about 5 to about 30 minutes, or about 5 to 20, 5 to 10, 10 to 30, 20 to 30 or 5 to 15 minutes, e.g. about 5, 10, 15, 20, 25 or 30 minutes. The acid may be then added directly to the reaction mixture. It may be added in excess, e.g. at least about 10 molar excess. The resulting mixture may be allowed to react for about 1 to about 10 minutes, or about 1 to 5, 5 to 10 or 3 to 8 minutes, e.g. about 1, 2, 3, 4, 5, 6, 7, 8, 9 or 10 minutes. It may be allowed to react at about the same temperature as the earlier reaction was conducted, or at a different temperature within the same range of temperatures. The resulting mixture may be neutralised, or approximately neutralised. It may be treated with a base, e.g. a hydroxide, in order to approximately neutralise the mixture. A buffer, e.g. a phosphate buffer, may be used to achieve the desired pH. The desired pH may be for example about 5 to about 8, or about 5 to 7, 5 to 6, 6 to 8, 7 to 8 or 6 to 7, e.g. about 5, 5.5, 6, 6.5, 7, 7.5 or 8. The resulting reaction mixture may be subjected to any suitable separation technique in order to separate the product. It may for example be applied to an HPLC column and the fraction containing the desired product isolated. That fraction may be dried and then the product may be formulated in a suitable buffer for further use. Alternatively, the desired product isolated from the HPLC column maybe further diluted with water and adsorbed onto a solid phase extraction column, washed with a suitable solvent and then reformulated in a suitable buffer. The suitable buffer may be suitable to maintain the resulting formulation at a pH of about 5 to about 8, or about 5 to 7, 5 to 6, 6 to 8, 7 to 8 or 6 to 7, e.g. about 5, 5.5, 6, 6.5, 7, 7.5 or 8.

Standard organic synthetic methods may be used to prepare the N-protected ester of the substituted S-propylhomocysteine having a leaving group on the propyl group. A suitable synthesis is outlined below, however it will be readily appreciated that other schemes may be used, and other sets of conditions, in order to reach this product.

Thus in a suitable synthesis homocystine (3,3′-amino-3,3′-dicarboxydipropyldisulfide) is N-protected using Boc₂O and mild base. The resulting N,N′-protected homocystine is then esterified using t-butyl 2,2,2-trichloroacetimidate, or other suitable t-butylating agent. Reduction of the product, e.g. using tributylphosphine, results in cleavage of the disulfide bond to produce the free N-protected and carboxyl protected thiol. This thiol is then treated with either a 3-bromo-1-halopropane or with 3-bromo-1-propanol, catalysed by a mild base such as carbonate. This step should be conducted under conditions (base, temperature) that do not lead to racemisation. In the event that a 3-bromo-1-halopropane is used, this results directly in an N-protected ester of the substituted propyl homocysteine having a halide leaving group on the propyl group. In the event that 3-bromo-1-propanol is used, the resulting alcohol is tosylated, e.g. using tosyl chloride and base, to provide an N-protected ester of the substituted 5-propylhomocysteine having a tosylate leaving group on the propyl group.

The process may additionally comprise purifying the ¹⁸F-radiolabelled propylhomocysteine. Suitably the purification may use chromatography, e.g. HPLC. As the product is radiolabelled, it is convenient to use a detector on the HPLC that is sensitive to radioactivity.

The compound of the present invention (i.e. the ¹⁸F-radiolabelled S-propylhomocysteine) may be used in tumour imaging. It may be used to make a composition for use in tumour imaging. In such applications it is commonly necessary to expose the compound to an aqueous environment. The inventors have surprisingly found that the ¹⁸F-radiolabelled S-propylhomocysteine is substantially more stable in such an environment than the corresponding S-ethyl homologue. This renders the S-propyl analogue more suitable for such applications than the S-ethyl analogue which has previously been investigated for such applications.

Described herein is the development and evaluation of a new analogue of [¹⁸F]FEHCys; S-(3-[¹⁸F]fluoropropyl)homocysteine ([¹⁸F]FPHCys). The chemical identification, characterisation, properties including stability and enantiomeric purity have not been described previously. Consequently, the biological properties of the individual D and L enantiomers of FPHCys or [¹⁸F]FPHCys have also not been described.

The inventors have developed a one-pot two-step radiolabelling method for the synthesis of [¹⁸F]FPHCys. In this process, the activated complexed ¹⁸F is heated with the N-protected ester of the substituted S-propylhomocysteine in a sealed vial followed by addition of reagents to induce hydrolysis and then transfer of the reaction mixture on to a HPLC for purification and formulation. This process can be undertaken by manual methods when the quantities of radioactivity used are low (typically less than 500 MBq). The inventors have developed and optimised the preparation of [¹⁸F]FPHCys on an automated synthesis module such as, but not limited, to the GE Tracerlab F_{X}F_{N} synthesis module such that significantly higher quantities of radioactivity (i.e. ¹⁸F-quantities exceeding 500 MBq, typically 37 GBq, but may be undertaken up to 400 GBq). Typically automated synthesis units such the GE Tracerlab F_{X}F_{N} synthesis module allow simple steps such as transfer of [¹⁸F]fluoride, reagents (such as potassium oxalate or other bases, Kryptofix™ 2.2.2, hydrochloric acid, solvents) and other materials through a system of valves reaction vessels and transfer lines. The one-pot two step synthesis, allows such processes to be more conveniently undertaken by such mechanical devices in a more efficient manner than would have been the case if a two pot synthesis were used. In this manner, the processes described in this work is more amenable to automated radiosynthesis and thus it ensures wider use and application than previous processes. In addition, the enantiomeric stability of this tracer has been assured and evaluated for potential biological and clinical applications. Suitably protected S-(3-tosyloxypropyl)homocysteine 1, S-(3-bromopropyl)homocysteine 2 and S-(3-chloropropyl)homocysteine 3 were synthesised as precursors and labelled by classical fluorine-18 nucleophilic substitution. Thus D and L isomers of S-(3-[¹⁸F]fluoropropyl)-L-homocysteine ([¹⁸F]FPHCys) were prepared separately by a one-pot two-step synthesis using GE Tracerlab F_{X}F_{N} synthesis module via the protected S-(3-tosyloxypropyl)homocysteine 1, S-(3-bromopropyl)homocysteine 2 and S-(3-chloropropyl)homocysteine 3 precursors. This radiolabelling involves a classical fluorine-18 nucleophilic substitution (performed at different temperature (50-100° C.) in acetonitrile using Kryptofix™ 222 (also known as K₂₂₂ or 4,7,13,16,21,24-hexaoxa-1,10-diazabicyclo[8.8.8]-hexacosane) and potassium carbonate or oxalate) followed by acid hydrolysis (performed at 100° C. for 5 min using hydrochloric acid). However, a racemisation of ([¹⁸F]FPHCys) was observed when this radiolabelling was performed in the presence of potassium carbonate. For this reason, potassium oxalate was used instead via the tosylate precursor 1, at 100° C. for 10 min followed by acid hydrolysis at 100° C. for 5 min. Under these conditions, [¹⁸F]FPHCys was isolated with an overall radiochemical yield of 30±5% decay-corrected (20±5% non decay-corrected), starting from, but not limited to, 18-37 GBq (500-1000 mCi) [¹⁸F]fluoride batch and with a radiochemical and enantiomeric purity>98% after 65 min of synthesis time including HPLC purification and the formulation.

The ultimate goal of the present work was an efficient one-pot synthesis of chemically stable and enantiomerically pure [¹⁸F]FPHCys that can be adopted to an automated synthesis process using, but not limited to, the automated synthesis units GE Tracerlab F_{X}F_{N} synthesis module. The simplified chemistry process, stability and its adaptation to automated synthesis ensures that the resultant [¹⁸F]-L-FPHCys and [¹⁸F]-D-FPHCys can be practically prepared in large quantities suitable for distribution and use by medical institutions. The synthesis of the required precursors, protected S-(3-tosyloxypropyl)homocysteine 1, S-(3-bromopropyl)homocysteine 2 and S-(3-chloropropyl)homocysteine 3, was achieved in four or five steps (FIG. 1) using classical chemistry methods. Protection of L-homocystine or D-homocystine with di-tert-butyl dicarbonate (P. Serafinowski, E. Dorland, K. R. Harrap, J. Balzarini, E. De Clercq, J. Med. Chem. 1992, 35, 4576-4583; J. Zhu, X. Hu, E. Dizin, D. Pei, J. am. Chem. Soc. 2003, 125, 13379-13381) followed by reaction of compound 4 with tert-butyl-2,2,2-trichloroacetimidate (J. McConathy, L. Martarello, E. J. Malveaux, V. M. Camp, N. E. Simpson, C. P. Simpson, G. D. Bowers, J. J. Olson, M. M. Goodman, j. Med. Chem. 2002, 45, 2240-2249; J. McConathy, L. Martarello, E. J. Malveaux, V. M. Camp, N. E. Simpson, C. P. Simpson, G. D. Bowers, Z. Zhang, J. J. Olson, M. M. Goodman, Nucl. Med. Biol. 2003, 30, 477-490) in dichloromethane at room temperature afforded quantitatively compound 5. Cleavage of the disulfide bond to form protected L-homocysteine 6 was achieved in 90% yield, using tributylphosphine in DMF (J. Zhu, X. Hu, E. Dizin, D. Pei, J. am. Chem. Soc. 2003, 125, 13379-13381; K. Yoshiizumi, F. Nakajima, R. Dobashi, N. Nishimura, S. Ikeda, Bioorg. Med. Chem. 2002, 10, 2445-2460; C. Lherbet, J. W. Keillor, Org. Biomol. Chem. 2004, 2, 238-245). Precursors 2-3 and compounds 7-8 were prepared in 77-96% yields by coupling 1-bromo-3-halopropane or 3-bromopropanol with thiol 6 in the presence of potassium carbonate in DMF at room temperature (K. Yoshiizumi, F. Nakajima, R. Dobashi, N. Nishimura, S. Ikeda, Bioorg. Med. Chem. 2002, 10, 2445-2460; C. Lherbet, J. W. Keillor, Org. Biomol. Chem. 2004, 2, 238-245). Protection of alcohol 8 with p-toluenesulfonyl chloride in presence of N,N,N′,N′-tetramethyl-1,6-hexanediamine in acetonitrile was achieved in 77% yield (Y. Yoshida, K. Shimonishi, Y. Sakakura, S. Okada, N. Aso, Y. Tanabe, Synthesis 1999, 9, 1633-1636). The overall yields of the tosylate, brominated and chlorinated-precursors 1-3 for the four or five steps were 66%, 69% and 83% respectively for the both enantiomers L and D.

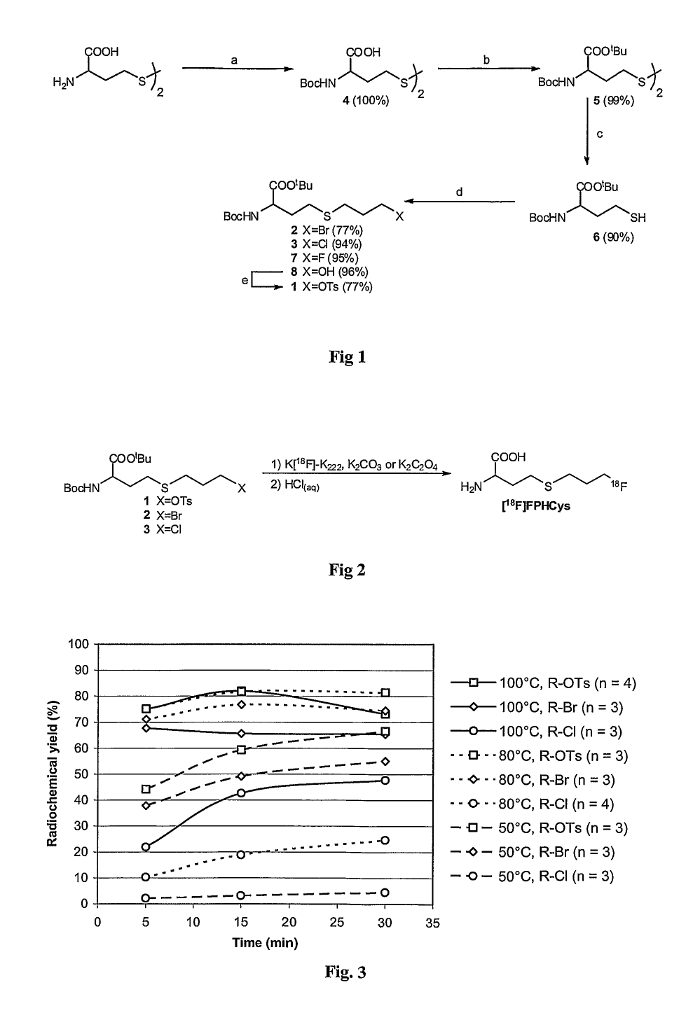

The radiosynthesis of [¹⁸F]-L-FPHCys from 1-3 (enantiomer L) was accomplished in one pot, two steps, by classical fluorine-18 nucleophilic substitution, followed by acid hydrolysis of the protecting groups (FIG. 2). Optimisation of the reaction conditions involved the reaction of [¹⁸F]-fluoride (370-555 MBq (10-15 mCi)) at different temperatures (50-100° C.) with 5 mg quantities of precursors 1-3 (10-14 μmol) in acetonitrile containing 10 mg K₂₂₂ (Kryptofix™ 222) and 2 mg potassium carbonate (FIG. 3). At 50° C. the tosylate precursor 1 showed higher reactivity (67% yield at 30 min), than the brominated and chlorinated analogue 2 and 3 (55% and 5% respectively). However, the radiochemical yield of the three precursors was significantly increased at 100° C. At this temperature, the chlorinated precursor 3 delivered a continuously increasing yield over the 30 min reaction until a maximum of 48% radiochemical yield was reached. Unlike the chlorinated precursor 2 which is stable up to 100° C. at high temperature, the tosylate precursor 1 showed significant decomposition at 100° C. after 15 min of reaction. At this same temperature, the radiochemical yield of the brominated precursor 2 was relatively stable from 67% at 5 min to 65% at 30 min. However, at 80° C., the brominated precursor 2 showed higher reactivity than at 100° C. (77% yield at 15 min). At this temperature, the tosylate precursor 1 showed a similar profile than at 100° C. but without decomposition after 15 min of reaction.

The radiosynthesis of [¹⁸F]-L-FPHCys was then completed by acid hydrolysis to remove both protecting groups. Hydrolysis of protected [¹⁸F]-L-FPHCys (generated from 1 for 10 min at 100° C. (77% yield)) was performed with 6 N HCl at 100° C. for 5 min. This reaction was then quenched with 6 N NaOH and diluted in phosphate buffer 1.5 M, pH 6. The resulting solution was purified by HPLC which confirmed complete hydrolysis and formation of [¹⁸F]FPHCys with an overall radiochemical yield of 47±5% decay-corrected and a radiochemical purity>98%. However, the racemisation of [¹⁸F]-L-FPHCys was observed ([¹⁸F]-L-FPHCys/[¹⁸F]-D-FPHCys, 75/25) due to basic condition of this labelling using potassium carbonate.

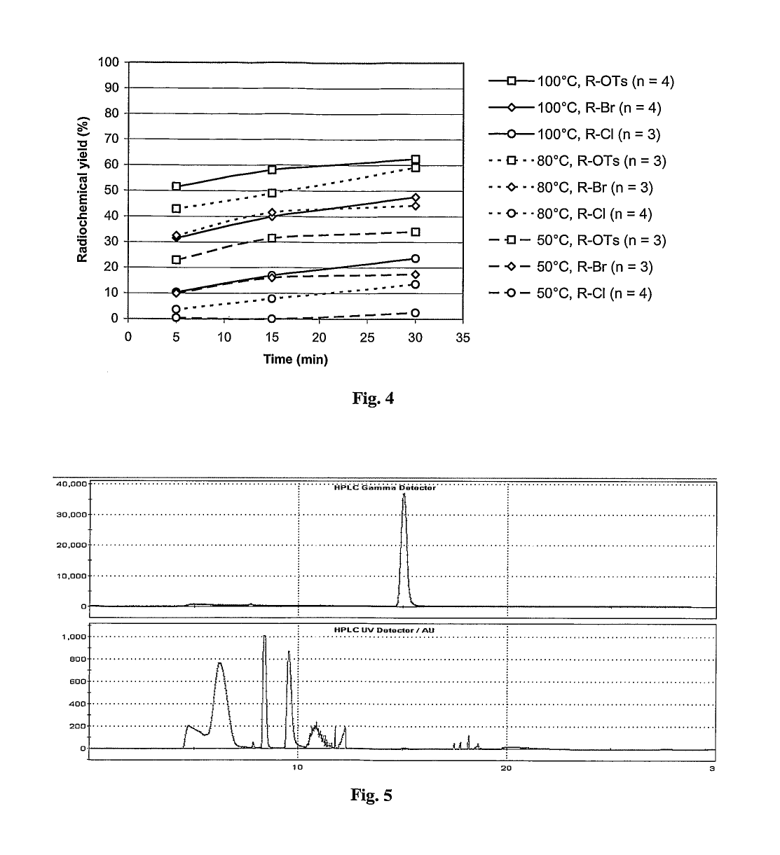

Subsequently, the radiosynthesis of [¹⁸F]-L-FPHCys was accomplished with 2.55 mg of potassium oxalate instead of potassium carbonate and 10 mg of K₂₂₂ at different temperatures (50-100° C.) with 5 mg quantities of precursors 1-3 (10-14 μmol) in acetonitrile (FIG. 4). In contrast to potassium carbonate, the fluorination with potassium oxalate showed a continuously increasing yield for the three precursors 1-3 at 50, 80 and 100° C. with higher reactivity at 100° C. However, all the radiochemical yields observed were lower when the fluorination was undertaken with potassium oxalate. The highest labelling efficiency observed with precursor 1 at 100° C. after 30 min was 62% yield with potassium oxalate compared to 82% with potassium carbonate after 15 min.

The radiosynthesis of [¹⁸F]-FPHCys was then completed by acid hydrolysis (6 N HCl) of the protected [¹⁸F]-L-FPHCys at 100° C. for 5 min. This reaction was then quenched with 6 N NaOH, diluted with phosphate buffer 1.5 M, pH 6 and the resultant solution purified by HPLC. [¹⁸F]FPHCys was obtained in an overall radiochemical yield of 38±5% decay-corrected and a radiochemical purity>98%. Under these conditions, [¹⁸F]-L-FPHCys was obtained in an enantiomeric purity exceeding 98%. The radiosynthesis of [¹⁸F]-D-FPHCys from 1-3 (enantiomer D) was accomplished using the same condition as that for [¹⁸F]-L-FPHCys with similar chemical and radiochemical purity.

In conclusion, [¹⁸F]-FPHCys and [¹⁸F]-D-FPHCys were prepared by a one-pot two-step synthesis, which has been fully automated on a GE Tracerlab F_{X}F_{N} synthesis module, using the tosylate precursor 1 (5 mg, 10 μmol) in acetonitrile at 100° C. for 10 min, in the presence of 2.55 mg of potassium oxalate and 10 mg of K₂₂₂ followed by acid hydrolysis of the protected [¹⁸F]FPHCys with 6N HCl at 100° C. for 5 min. Typically, 4.1-7.8 GBq (110-210 mCi) of [¹⁸F]FPHCys was isolated with an overall radiochemical yield of 30±5% decay-corrected (20±5% non decay-corrected) within 65 min including HPLC purification and the formulation, starting from 18-37 GBq (500-1000 mCi) [¹⁸F]fluoride batch and with a radiochemical and enantiomeric purity>98%.

### EXAMPLE

### Experimental

General. The reagents and solvents used in this preparation were purchased from Lancaster, Fluka or Sigma-Aldrich. All chemicals and solvents were used without further purification.

All melting points were determined in open capillary tubes using a SRS Optimet automated melting point system, MPA 100 and are uncorrected. ¹H and ¹³C NMR spectra were measured on a Brucker DPX 400, at 400 MHz (¹H) and 100 MHz (¹³C) in an appropriate deuterated solvent (CDCl₃, CD₃OD, D₂O). Chemical shifts are reported as parts per million (δ) relative to tetramethylsilane (TMS, 0.00 ppm), which was used as an internal standard. Coupling constants are given in Hz and coupling constants are abbreviated as: s (singlet), d (doublet), t (triplet), q (quadruplet), qu (quintuplet), m (mutiplet), and dt (doublet of triplet). Low-resolution mass spectrometry (LRMS) was performed on a Waters Micromass ZQ Quadrupole Mass Spectrometer whereas High-resolution mass spectrometry (HRMS) was performed using a Micromass Qtof Ultima or AutoSpec TOF. TLCs were run on pre-coated aluminum plates of silica gel 60F₂₅₄ (Merck) and Rf were established using an UV-lamp at 254 nm. Column chromatography was undertaken on Merck 60 silica gel (40-63 μm) columns.

[¹⁸F]HF was produced on a Cyclotron via the ¹⁸O(p, n)¹⁸F. The radiolabelling was performed on a GE Tracerlab F_{X}F_{N} synthesis module. The intermediate product labelled with fluorine-18 was analysed by HPLC, a Waters 510 pump, a Linear UVIS detector (λ=210 nm) in series with a Berthhold β⁺-flow detector, on a Phenomenex Bondclone C18 column (300×7.8 mm, 10 μm) at 2 mL/min with CH₃CN/H₂O (75:25, v/v) as the mobile phase. The final product labelled with fluorine-18 was purified by HPLC, consisting of a Sykam S-1021 pump, a Knauer K-2001 UV detector (λ=220 nm) in series with a Berthhold β⁺-flow detector, on a Waters AtlantisC18 column (250×10 mm, 5 μm) at 3 mL/min with EtOH/Phosphate buffer, pH 6, 0.15 M (5:95, v/v) as the mobile phase. Quality control analysis of the hydrolysed radioligand was performed on a Varian 9002 pump, a Linear UVIS detector (λ=226 nm) in series with an Ortec ACE Mate β⁺-flow detector on a Phenomenex Gemini column (150×4.6 mm, 5 μm) at 1 mL/min with Phosphate buffer, pH 11.5, 0.05 M as the mobile phase for chemical and radiochemical purity and on a Phenomenex Chirex D Penicilamine column (150×4.6 mm, 5 μm) at 1 mL/min with isopropanol/CuSO_{4 aq}, 2 nM (1:9, v/v) as the mobile phase for enantiomeric purity. The identity of the labelled compound was confirmed by co-injection with the authentic compound on HPLC. Radioactivity was measured with a Capintec R15C dose calibrator.

### Chemistry and Radiochemistry

N,N′-Di(tert-butoxycarbonyl)homocystine (4). Prepared by the published procedure (J. Zhu, X. Hu, E. Dizin, D. Pei, J. am. Chem. Soc. 2003, 125, 13379-13381). The following additional analytical data was obtained: mp 158-159° C.; ¹³C NMR (CD₃OD) δ 175.68, 158.16, 80.69, 53.72, 35.80, 32.54, 28.82; LRMS: ES(+ve) m/z 469 (M+1); HRMS: ES(+ve) Calcd for C₁₈H₃₂N₂O₈S₂ (M+1) 469.1703, found 469.1678.

Di(tert-butyl) N,N′-bis(tert-butoxycarbonyl)homocystinate (5). Tert-butyl-2,2,2-trichloroacetimidate (11.0 g, 47.77 mmol) was added to a solution of 4 (4.47 g, 9.55 mmol) in dichloromethane (39 mL) under nitrogen. After overnight stirring at room temperature, the solvent was removed under reduced pressure and the crude residue purified by chromatography on silica gel (heptanes/ethyl acetate 80:20) to give 5 (5.50 g, 99%) as a white solid. mp 66-68° C.; ¹H NMR and ¹³C NMR identical to publish data (J. Zhu, X. Hu, E. Dizin, D. Pei, J. am. Chem. Soc. 2003, 125, 13379-13381); LRMS: ES(+ve) m/z 581 (M+1); HRMS: ES(+ve) Calcd for C₂₆H₄₈N₂O₈S₂ (M+1) 581.2938, found 581.2930.

tert-Butyl N-(tert-butoxycarbonyl)homocysteinate (6). To a solution of 5 (3.20 g, 5.51 mmol) in DMF (45 mL) was added water (4 mL) and tributylphosphine (1.26 g, 6.06 mmol) and the mixture stirred under nitrogen at room temperature overnight. The reaction mixture was quenched with water (500 mL) and the aqueous phase thoroughly extracted with ethyl acetate (3×240 mL). The combined organic layers were washed with brine (2×150 mL), dried (MgSO₄) and the solvents evaporated. The crude residue was purified by chromatography on silica gel (heptanes/ethyl acetate 90:10) to give 6 (2.90 g, 90%) as a colourless oil. ¹H NMR and ¹³C NMR identical to publish data (J. Zhu, X. Hu, E. Dizin, D. Pei, J. am. Chem. Soc. 2003, 125, 13379-13381); LRMS: ES(+ve) m/z 292 (M+1); HRMS: ES(+ve) Calcd for C₁₃H₂₅NO₄S (M+1) 292.1579, found 292.1583.

tert-Butyl N-(tert-butoxycarbonyl)-S-(3-fluoropropyl)homocysteinate (7). To a solution of 6 (445 mg, 1.53 mmol) in DMF (6.7 mL) was added 1-bromo-3-fluoropropane (326 mg, 2.29 mmol) followed by potassium carbonate (422 mg, 3.05 mmol) and the resultant white solution stirred for 24 h at room temperature under nitrogen. The reaction mixture was diluted with water (90 mL) and extracted with ethyl acetate (3×45 mL). The combined organic layers were washed with water (90 mL), dried (MgSO₄) and the solvents evaporated. The crude residue was purified by chromatography on silica gel (heptanes/ethyl acetate 90:10) to give 7 (504 mg, 95%) as a yellow oil. ¹H NMR (CDCl₃) δ 5.15-5.05 (m, 1H), 4.53 (dt, J=47.1 Hz, J=5.8 Hz, 2H), 4.35-4.20 (m, 1H), 2.64 (t, J=7.3 Hz, 2H), 2.60-2.50 (m, 2H), 2.15-2.03 (m, 1H), 2.03-1.80 (m, 3H), 1.47 (s, 9H), 1.44 (s, 9H); ¹³C NMR (CDCl₃) δ 171.45, 155.48, 82.44 (J=165.6 Hz), 82.35, 79.95, 53.55, 33.28, 30.54 (J=20.7 Hz), 28.45, 28.15, 28.08, 27.90 (J=5.4 Hz); LRMS: ES(+ve) m/z 374 (M+Na); HRMS: ES(+ve) Calcd for C₁₆H₃₀NO₄SF (M+1) 352.1958, found 352.1970.

tert-Butyl N-(tert-butoxycarbonyl)-S-(3-hydroxypropyl)homocysteinate (8). This compound was synthesized as described for 7 from 6 (6 g, 20.61 mmol) and 3-bromopropanol (4.43 mg, 30.92 mmol). The crude residue was purified by chromatography on silica gel (heptanes/ethyl acetate 70:30) to give 8 (6.91 mg, 96%) as a yellow oil. ¹H NMR (CDCl₃) δ 5.12-5.05 (m, 1H), 4.35-4.20 (m, 1H), 3.80-3.70 (m, 2H), 2.64 (t, J=6.7 Hz, 2H), 2.60-2.50 (m, 2H), 2.15-2.05 (m, 1H), 1.95-1.80 (m, 2H), 1.83 (qu, J=6.7 Hz, 2H), 1.47 (s, 9H), 1.44 (s, 9H); ¹³C NMR (CDCl₃) δ 172.56, 156.56, 82.35, 81.06, 61.62, 54.51, 33.18, 31.98, 28.80, 28.47, 28.16, 28.02; LRMS: ES(+ve) m/z 372 (M+Na); HRMS: ES(+ve) Calcd for C₁₆H₃₁NO₅S (M+1) 350.2001, found 350.2017.

tert-Butyl S-(3-bromopropyl)-N-(tert-butoxycarbonyl)homocysteinate (2). This compound was synthesized as described for 7 from 6 (200 mg, 0.68 mmol) and 1,3-dibromopropane (420 mg, 2.06 mmol). The crude residue was purified by chromatography on silica gel (heptanes/ethyl acetate 90:10) to give 2 (215 mg, 77%) as a yellow oil. ¹H NMR (CDCl₃) δ 5.15-5.05 (m, 1H), 4.35-4.20 (m, 1H), 3.51 (t, J=6.7 Hz, 2H), 2.67 (t, J=6.7 Hz, 2H), 2.60-2.50 (m, 2H), 2.15-2.05 (m, 1H), 2.10-2.05 (qu, J=6.7 Hz, 2H), 1.95-1.80 (m, 1H), 1.47 (s, 9H), 1.44 (s, 9H); ¹³C NMR (CDCl₃) δ 171.43, 155.47, 82.37, 79.98, 53.53, 33.34, 32.31, 32.21, 30.36, 28.47, 28.16, 28.02; LRMS: ES(+ve) m/z 412 (M[Br⁷⁹]+1), 414 (M[Br⁸¹]+1); HRMS: ES(+ve) Calcd for C₁₅H₂₈NO₄SBr (M[Br⁷⁹]) 412.1157, found 412.1165.

tert-Butyl N-(tert-butoxycarbonyl)-S-(3-chloropropyl)homocysteinate (3). This compound was synthesized as described for 7 from 6 (200 mg, 0.68 mmol) and 1-bromo-3-chloroethane (327 mg, 2.06 mmol). The crude residue was purified by chromatography on silica gel (heptanes/ethyl acetate 90:10) to give 3 (236 mg, 94%) as a yellow oil. ¹H NMR (CDCl₃) δ 5.15-5.05 (m, 1H), 4.35-4.20 (m, 1H), 3.64 (t, J=6.7 Hz, 2H), 2.67 (t, J=6.7 Hz, 2H), 2.60-2.50 (m, 2H), 2.15-2.05 (m, 1H), 2.02 (qu, J=6.7 Hz, 2H), 1.95-1.80 (m, 1H), 1.47 (s, 9H), 1.44 (s, 9H); ¹³C NMR (CDCl₃) δ 171.43, 155.48, 82.36, 79.97, 53.53, 43.54, 33.32, 32.25, 29.16, 28.46, 28.16, 28.03; LRMS: ES(+ve) m/z 368 (M[Cl³⁵]+1), 370 (M[Cl³⁷]+1); HRMS: ES(+ve) Calcd for C₁₆H₃₀NO₄SCl (M[Cl³⁵]+1) 368.1662, found 368.1677.

tert-Butyl N-(tert-butoxycarbonyl)-S-(3-tosyloxypropyl)homocysteinate (1). To a solution of 8 (2.85 g, 8.20 mmol) in acetonitrile (115 mL) was added N,N,N′,N′-tetramethyl-1,6-hexanediamine, (2.84 g, 16.14 mmol) followed by p-toluenesulfonyl chloride (2.39 g, 12.30 mmol) and the reaction mixture stirred for 4 h at room temperature under nitrogen. The reaction mixture was diluted with water (340 mL) and extracted with ethyl acetate (3×340 mL). The combined organic layers were washed with brine (340 mL), dried (MgSO₄) and the solvents evaporated. The crude residue was purified by chromatography on silica gel (heptanes/ethyl acetate 80:20) to give 1 (3.19 g, 77%) as a yellow oil. ¹H NMR (CDCl₃) δ 7.78 (d, J=8.2 Hz, 2H), 7.34 (d, J=8.2 Hz, 2H), 5.15-5.05 (m, 1H), 4.30-4.15 (m, 1H), 4.12 (t, J=6.7 Hz, 2H), 2.53 (t, J=6.7 Hz, 2H), 2.50-2.40 (m, 2H), 2.45 (s, 3H), 2.10-1.95 (m, 1H), 1.90 (qu, J=6.7 Hz, 2H), 1.90-1.75 (m, 2H), 1.46 (s, 9H), 1.43 (s, 9H); ¹³C NMR (CDCl₃) δ 171.37, 155.47, 144.97, 133.15, 130.03, 128.05, 82.37, 79.97, 68.88, 53.51, 33.19, 29.82, 28.45, 28.14, 27.99, 27.97, 21.78; LRMS: ES(+ve) m/z 526 (M+Na); HRMS: ES(+ve) Calcd for C₂₃H₃₇NO₇S₂ (M+1) 504.2090, found 504.2105.

S-(3-Fluoropropyl)homocysteine hydrochloride (FPHCys). To a solution of the protected amino acid 7 (63 mg) in THF (530 μL) was added 6 N hydrochloric acid (1.07 mL) and the reaction mixture stirred at room temperature for 3 h. The solvent was removed under reduced pressure, and the resulting solid washed with ethyl acetate (3×7 mL), dried under vacuum to give FPHCys (38 mg, 91%) as a white solid. ¹H NMR (D₂O) δ 4.64 (dt, J=47.1 Hz, J=5.8 Hz, 2H), 4.17 (t, J=6.2 Hz, 1H), 2.77 (t, J=7.3 Hz, 2H), 2.75 (t, J=7.0 Hz, 2H), 2.35-2.15 (m, 2H), 2.10-1.95 (m, 2H); ¹³C NMR (D₂O) δ 173.52, 84.88 (d, J=158.7 Hz), 53.52, 30.81, 30.69 (d, J=19.9 Hz), 27.84 (d, J=5.4 Hz), 27.64; LRMS: ES(+ve) m/z 196 (M+1); HRMS: ES(+ve) Calcd for C₇H₁₄NO₂SF (M+1) 196.0808, found 196.0814.

Radiosynthesis of S-(3-[¹⁸F]fluoropropyl)homocysteine ([¹⁸F]FPHCys). On GE Tracerlab F_{X}F_{N} synthesis module, an aqueous [¹⁸F]fluoride solution (18-37 GBq (500-1000 mCi)) was loaded on a QMA cartridge firstly activated with potassium oxalate (10 mL, 0.11 M). The concentrate [¹⁸F]fluoride was eluted with a solution of K₂C₂O₄/K₂CO₃ (2.55 mg/50 μg, 200 μL in H₂O) and K₂₂₂ (10 mg, 800 μL in CH₃CN) in the reactor. The solvent was evaporated under a stream of helium at 70° C. under vacuum for 7 min and then at 120° C. under vacuum only for 5 min. To the activated K₂₂₂/potassium [¹⁸F]fluoride was added the tosylate precursor 1 (5 mg, 10 μmol) in acetonitrile (2 mL) and the mixture heated at 100° C. for 10 min before the addition 6 N HCl (500 μL). After 5 min at 100° C., 6 N NaOH (500 μL) and phosphate buffer pH 6 (1 mL, 1.5 M) were added and the resulting solution was directly purified on a semi-preparative HPLC column (AtlantisC18 column (250×10 mm, 5 μm); mobile phase ethanol/phosphate buffer, pH 6, 0.15 M (5:95, v/v); flow rate 3 mL/min; λ=220 nm). The fraction containing the labelled product [¹⁸F]FPHCys was collected between 15 and 16 min and formulated in a sterile buffer pH 7 for cell studies and in saline for animal studies. Routinely, [¹⁸F]FPHCys was ready for injection in about 65 min. Typically, 4.1-7.8 GBq (110-210 mCi) of [¹⁸F]FPHCys was isolated with an overall radiochemical yield of 30±5% decay-corrected (20±5% non decay-corrected), starting from 18-37 GBq (500-1000 mCi) [¹⁸F]fluoride batch and with a radiochemical purity and enantiomeric purity>98%.

### Conclusion

The inventors have developed a one-pot two-step synthesis of [¹⁸F]FPHCys via protected S-(3-tosyloxypropyl)homocysteine 1, S-(3-bromopropyl)homocysteine 2 and S-(3-chloropropyl)homocysteine 3 precursors. The tosylate derivative 1 gave higher radiochemical yields at 100° C. However, a racemisation of ([¹⁸F]FPHCys) was observed when this radiolabelling was performed in presence of potassium carbonate. For this reasons, potassium oxalate was used instead of potassium carbonate. Finally, [18F]-L-FPHCys and [¹⁸F]-D-FPHCys were synthesised, via the tosylate precursor 1, at 100° C. for 10 min followed by acid hydrolysis at 100° C. for 5 min. In this condition, [¹⁸F]FPHCys was isolated with an overall radiochemical yield of 30±5% decay-corrected (20±5% non decay-corrected), and with a radiochemical and enantiomeric purity>98% after 65 min of synthesis time including HPLC purification and the formulation. Consequently, this one pot, two step synthesis offers the opportunity for the preparation of either enantiomer of the amino acid [¹⁸F]FPHCys with high enantiomeric purity using automated synthesis and provides scope for routine use of these enantiomers in the clinic.

### Stability and Enantiomeric Purity of [¹⁸F]-L-FPHCys and [¹⁸F]-D-FPHCys

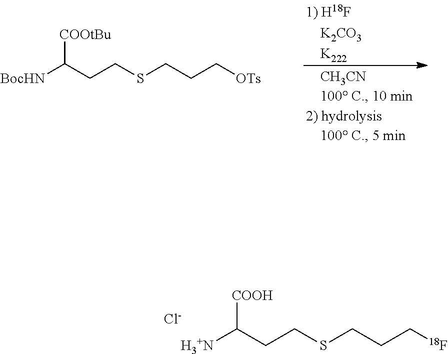

### 1) Radiosynthesis

[¹⁸F]-L-FPHCys and [¹⁸F]-D-FPHCys were synthesised using the GETracerlab Fx_{FN} module according to the above scheme. The synthesis was described above.

### HPLC Purification of [¹⁸F]-L-FPHCys and [¹⁸F]-D-FPHCys Before QC Analysis:

The purification of [¹⁸F]-L-FPHCys and [¹⁸F]-D-FPHCys was performed on the GE Tracerlab, using an Atlantis C18 250×10 mm, 5 μm, eluting with 5% EtOH/95% Phosphate Buffer, pH 6, 0.15 M at 2.5 mL/min, λ=220 nm. A typical chromatogram is shown in FIG. 5. The fraction containing the labelled product [¹⁸F]FPHCys was collected between 15 and 16 min. [¹⁸F]FPHCys was isolated with an overall radiochemical yield of 47±5% decay-corrected in presence of potassium carbonate and with an overall radiochemical yield of 30±5% decay-corrected in presence of potassium oxalate.

### 2) QC Analysis:

### 2.1) Enantiomeric Analysis

The enantiomeric analysis was performed on a Chirex D Penicilamine 150×4.6 mm, eluting with 10% isopropanol/90% CuSO_{4 aq}, 2 nM at 1 mL/min, λ=226 nm.

### 2.1.1) [¹⁸F]-L-FPHCys

### With Potassium Carbonate (K₂CO₃)

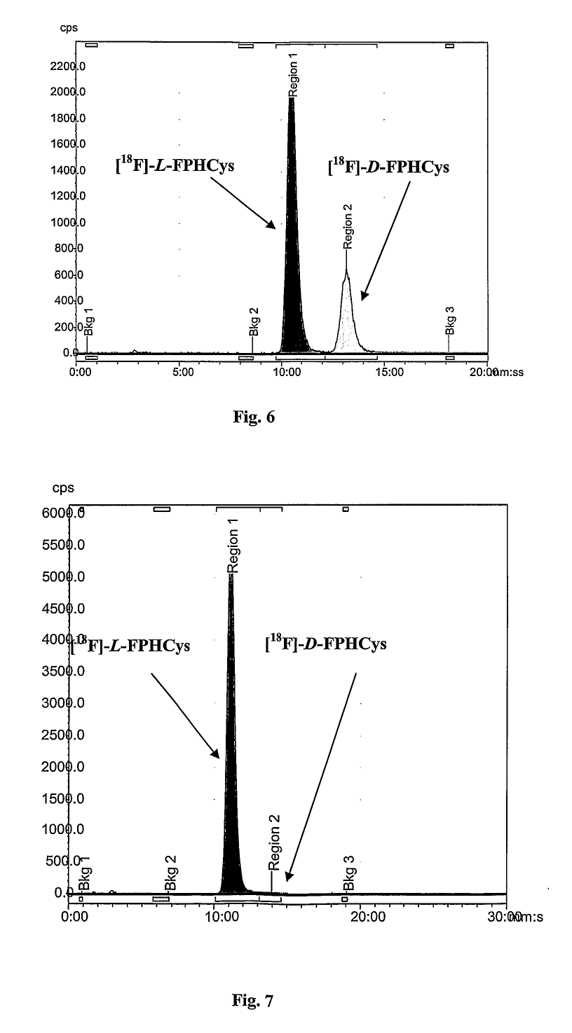

Firstly, the radiosynthesis of [¹⁸F]-L-FPHCys was accomplished in presence of potassium carbonate. The F18 chromatogram is shown in FIG. 6 and data is shown in the table below.

| Retention | Height | Area | % Total | % ROI |
|---|---|---|---|---|
| (mm:ss) | (cps) | (cps) | (%) | (%) |
| 0:32 | 6.0 | | | |
| 8:37 | 6.3 | | | |
| 10:31 | 2300.5 | 73308.5 | 73.85 | (L) 74.10 |
| 13:09 | 670.3 | 25617.8 | 25.81 | (D) 25.90 |
| 18:08 | 9.0 | | | |
| | | 98926.3 | 99.66 | 100.00 |

In the presence of potassium carbonate, a racemisation [¹⁸F]-L-FPHCys was observed. It was found that the ratio of ([¹⁸F]-L-FPHCys/[¹⁸F]-D-FPHCys, was 75/25) due to the basic condition of this labelling process.

To reduce the racemisation of [¹⁸F]-L-FPHCys, the radiosynthesis of [¹⁸F]-L-FPHCys was accomplished with potassium oxalate a weaker base instead of potassium carbonate.

### With Potassium Oxalate (K₂C₂O₄)

The F18 chromatogram of the resulting product is shown in FIG. 7 and data is shown in the table below.

| Retention | Height | Area | % Total | % ROI |
|---|---|---|---|---|
| (mm:ss) | (cps) | (cps) | (%) | (%) |
| 0:54 | 5.0 | | | |
| 6:46 | 9.4 | | | |
| 11:03 | 5905.2 | 214668.6 | 99.77 | (L) 99.52 |
| 13:57 | 26.2 | 1025.5 | 0.48 | (D) 0.48 |
| 19:05 | 11.3 | | | |
| | | 215694.1 | 100.25 | 100.00 |

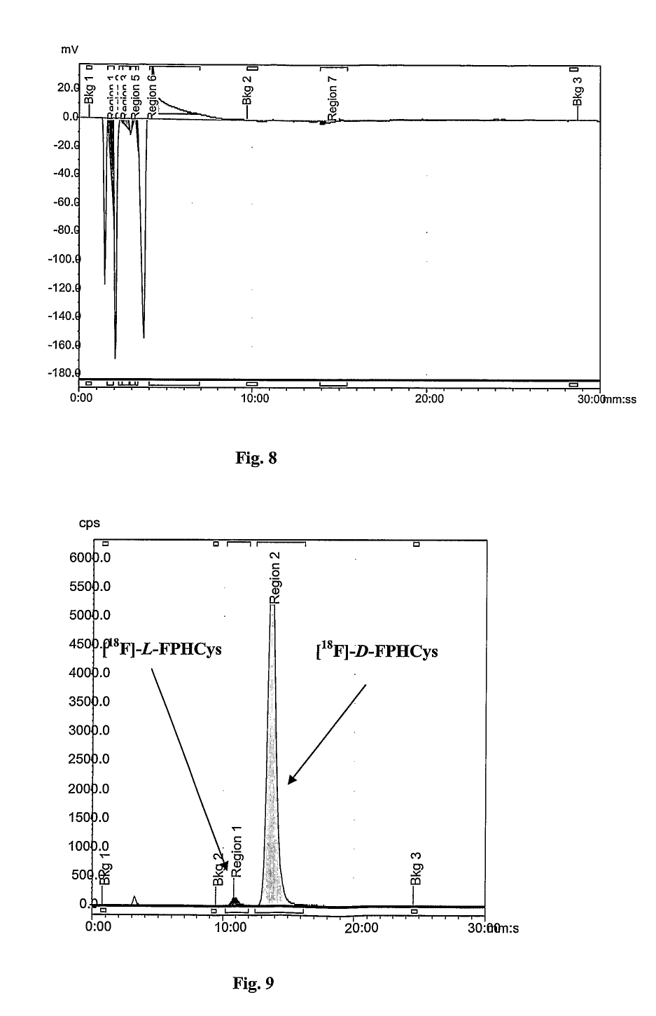

The UV chromatogram (226 nm) of the product is shown in FIG. 8 and data is shown in the table below.

| Retention | Height | Area | % Total | % ROI |
|---|---|---|---|---|
| (mm:ss) | (mV) | (mV) | (%) | (%) |
| 0:32 | 0.0 | | | |
| 1:51 | 29.8 | 710.7 | N/A | 32.11 |
| 2:19 | 3.1 | 30.4 | N/A | 1.37 |
| 2:38 | 5.5 | 179.4 | N/A | 8.11 |
| 3:04 | 2.9 | 67.3 | N/A | 3.04 |
| 3:14 | 0.7 | 59.9 | N/A | 2.71 |
| 4:11 | 36.2 | 1070.7 | N/A | 48.37 |
| 9:40 | −1.3 | | | |
| 14:35 | 1.2 | 94.9 | N/A | 4.29 |
| 28:41 | −0.8 | | | |
| | | 2213.2 | N/A | 100.00 |

Under these conditions [¹⁸F]-L-FPHCys was obtained with an enantiomeric purity over 99%. This same conditions were applied for the synthesis of [¹⁸F]-D-FPHCys.

### 2.1.2) [¹⁸F]-D-FPHCys

### With Potassium Oxalate (K₂C₂O₄)

The F18 chromatogram of the resulting product is shown in FIG. 9 and data is shown in the table below.

| Retention | Height | Area | % Total | % ROI |
|---|---|---|---|---|
| (mm:ss) | (cps) | (cps) | (%) | (%) |
| 0:46 | 3.0 | | | |
| 9:25 | 3.2 | | | |
| 10:51 | 141.3 | 4638.2 | 1.64 | (L) 1.66 |
| 13:38 | 6083.8 | 274243.7 | 96.84 | (D) 98.34 |
| 24:31 | 8.2 | | | |
| | | 278881.8 | 98.48 | 100.00 |

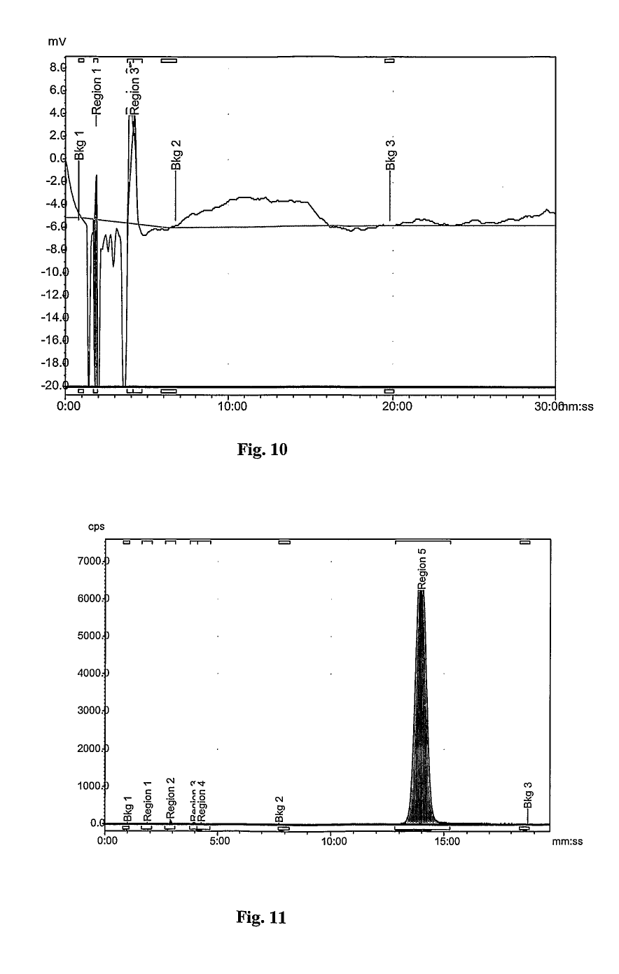

The UV chromatogram (226 nm) of the product is shown in FIG. 10 and data is shown in the table below.

| Retention | Height | Area | % Total | % ROI |
|---|---|---|---|---|
| (mm:ss) | (mV) | (mV) | (%) | (%) |
| 0:49 | −4.9 | | | |
| 1:51 | 4.5 | 332.8 | 64.39 | 77.71 |
| 3:54 | 9.1 | 136.0 | 26.32 | 31.76 |
| 4:11 | 4.0 | −40.5 | −7.84 | −9.46 |
| 6:44 | −5.8 | | | |
| 19:49 | −5.8 | | | |
| | | 428.3 | 82.87 | 100.00 |

The enantiomeric purity of [¹⁸F]-D-FPHCys using the potassium oxalate was obtained with an enantiomeric purity>98%. The enantiomeric purity of [¹⁸F]-D-FPHCys was less that [¹⁸F]-L-FPHCys due to the enantiomeric purity of the precursor. The precursor was synthesised with D-homocystine as starting material with an enantiomeric purity>98%.

### 2.2) Radiochemical Purity and Stability

The stability of [¹⁸F]-L-FPHCys and [¹⁸F]-D-FPHCys formulated in each of the following solutions:
1) 5% EtOH/95% phosphate buffer, pH 6, 0.15 M (HLPC solvent)
2) saline
3) Dulbecco's phosphate buffered saline, pH 7
4) Phosphate buffer, pH 7.
   were analysed by HPLC using a Gemini 150×4.6 mm, 5 μm, eluting with 100% Phosphate Buffer, pH 11.5, 0.05 M at 1 mL/min, λ=226 nm.

### 5% EtOH/95% Phosphate Buffer, pH 6, 0.15 M

(HLPC Solvent): t=0

The F18 chromatogram of the product is shown in FIG. 11 and data is shown in the table below.

| Retention | Height | Area | % Total | % ROI |
|---|---|---|---|---|
| (mm:ss) | (cps) | (cps) | (%) | (%) |
| 0:59 | 7.0 | | | |
| 1:52 | 13.2 | 88.8 | 0.04 | 0.04 |
| 2:53 | 96.7 | 360.0 | 0.15 | 0.16 |
| 3:57 | 18.5 | 80.6 | 0.03 | 0.03 |
| 4:17 | 13.4 | 68.1 | 0.03 | 0.03 |
| 7:45 | 8.4 | | | |
| 13:58 | 7293.5 | 231544.3 | 99.38 | 99.74 |
| 18:42 | 15.8 | | | |
| | | 232141.9 | 99.64 | 100.00 |

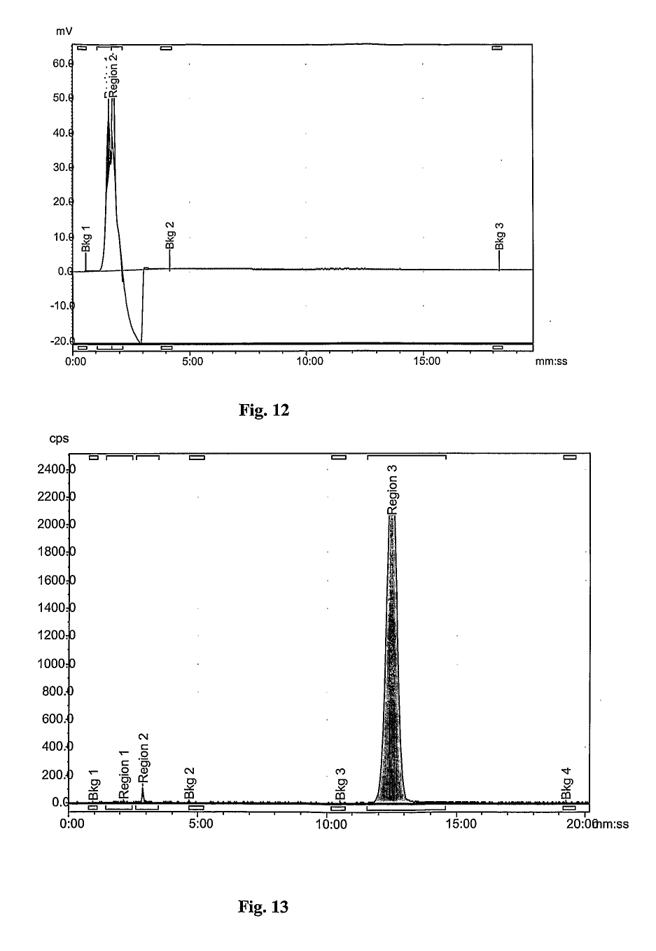

The UV chromatogram (226 nm) of the product is shown in FIG. 12 and data is shown in the table below.

| Retention | Height | Area | % Total | % ROI |
|---|---|---|---|---|
| (mm:ss) | (mV) | (mV) | (%) | (%) |
| 0:33 | 0.1 | | | |
| 1:32 | 44.4 | −31.1 | −6.80 | −208.03 |
| 1:44 | 63.1 | 46.1 | 10.07 | 308.03 |
| 4:09 | 0.9 | | | |
| 18:15 | 0.5 | | | |
| | | 15.0 | 3.27 | 100.00 |

### 5% EtOH/95% Phosphate Buffer, pH 6, 0.15 M

(HLPC Solvent): t=4 h

The F18 chromatogram of the product is shown in FIG. 13 and data is shown in the table below.

| Retention | Height | Area | % Total | % ROI |
|---|---|---|---|---|
| (mm:ss) | (cps) | (cps) | (%) | (%) |
| 0:56 | 4.0 | | | |
| 2:08 | 10.1 | 115.2 | 0.16 | 0.17 |
| 2:51 | 122.2 | 536.0 | 0.77 | 0.77 |
| 4:39 | 6.2 | | | |
| 10:32 | 11.8 | | | |
| 12:29 | 2414.7 | 68636.9 | 98.10 | 99.06 |
| 19:16 | 12.4 | | | |
| | | 69288.1 | 99.03 | 100.00 |

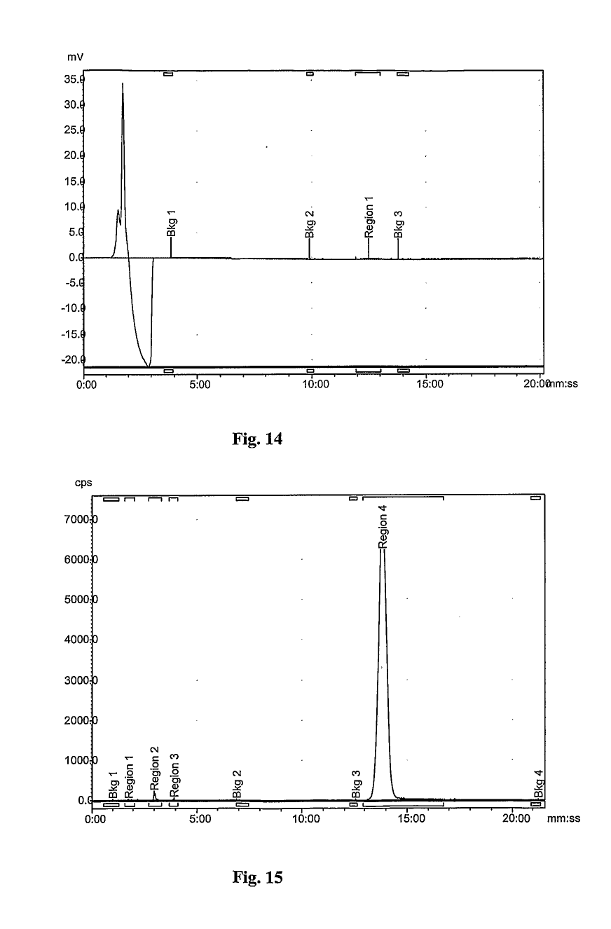

The UV chromatogram (226 nm) of the product is shown in FIG. 14 and data is shown in the table below.

| Retention | Height | Area | % Total | % ROI |
|---|---|---|---|---|
| (mm:ss) | (mV) | (mV) | (%) | (%) |
| 3:52 | 0.1 | | | |
| 9:55 | −0.2 | | | |
| 12:30 | −0.2 | 0.8 | N/A | 100.00 |
| 13:47 | −0.2 | | | |
| | | 0.8 | N/A | 100.00 |

The QC analysis revealed that both enantiomers in each of the formulations were stable with radiochemical purity ranging from 99.7% to 99.0% over a period of 4 h in 5% EtOH/95% phosphate buffer, pH 6, 0.15 M (HLPC solvent).

Saline: t=0

The F18 chromatogram of the product is shown in FIG. 15 and data is shown in the table below.

| Retention | Height | Area | % Total | % ROI |
|---|---|---|---|---|
| (mm:ss) | (cps) | (cps) | (%) | (%) |
| 1:00 | 9.1 | | | |
| 1:45 | 21.2 | 119.7 | 0.06 | 0.06 |
| 3:00 | 199.7 | 1099.4 | 0.57 | 0.57 |
| 3:55 | 25.6 | 122.0 | 0.06 | 0.06 |
| 6:54 | 10.4 | | | |
| 12:32 | 7.6 | | | |
| 13:48 | 7302.2 | 190924.8 | 98.81 | 99.30 |
| 21:14 | 13.7 | | | |
| | | 192266.0 | 99.50 | 100.00 |

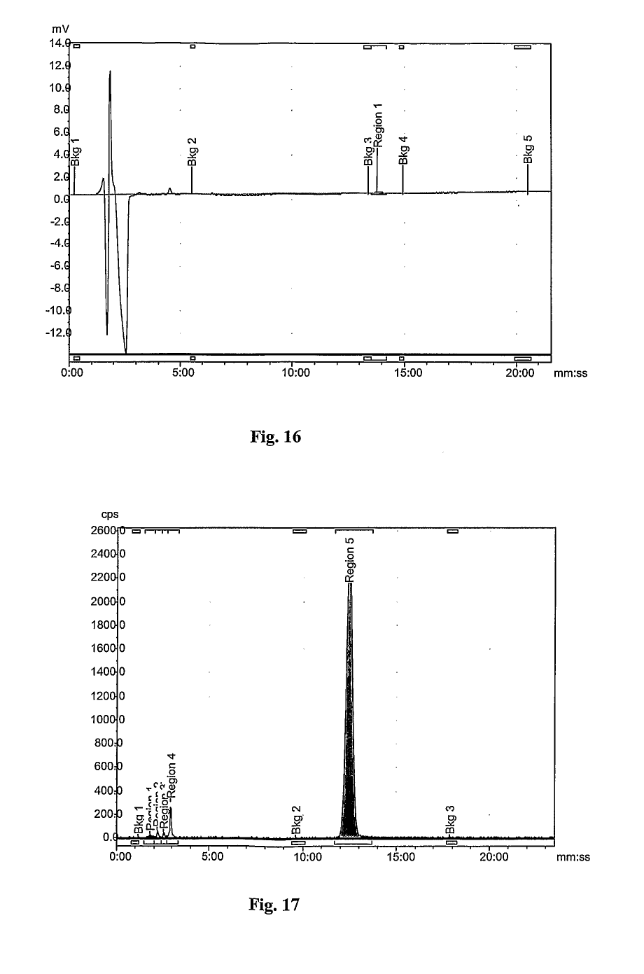

The UV chromatogram (226 nm) of the product is shown in FIG. 16 and data is shown in the table below.

| Retention | Height | Area | % Total | % ROI |
|---|---|---|---|---|
| (mm:ss) | (mV) | (mV) | (%) | (%) |
| 0:12 | 0.5 | | | |
| 5:31 | 0.5 | | | |
| 13:22 | 0.6 | | | |
| 13:46 | 0.7 | 1.0 | N/A | 100.00 |
| 14:55 | 0.7 | | | |
| 20:31 | 0.8 | | | |
| | | 1.0 | N/A | 100.00 |

Saline: t=4 h

The F18 chromatogram of the product is shown in FIG. 17 and data is shown in the table below.

| Retention | Height | Area | % Total | % ROI |
|---|---|---|---|---|
| (mm:ss) | (cps) | (cps) | (%) | (%) |
| 1:09 | 8.1 | | | |
| 1:46 | 32.4 | 321.7 | 0.53 | 0.53 |
| 2:10 | 67.9 | 402.6 | 0.66 | 0.67 |
| 2:31 | 51.8 | 283.7 | 0.47 | 0.47 |
| 2:54 | 310.6 | 1864.2 | 3.07 | 3.09 |
| 9:38 | 8.5 | | | |
| 12:25 | 2525.0 | 57369.1 | 94.55 | 95.23 |
| 17:52 | 12.3 | | | |
| | | 60241.3 | 99.28 | 100.00 |

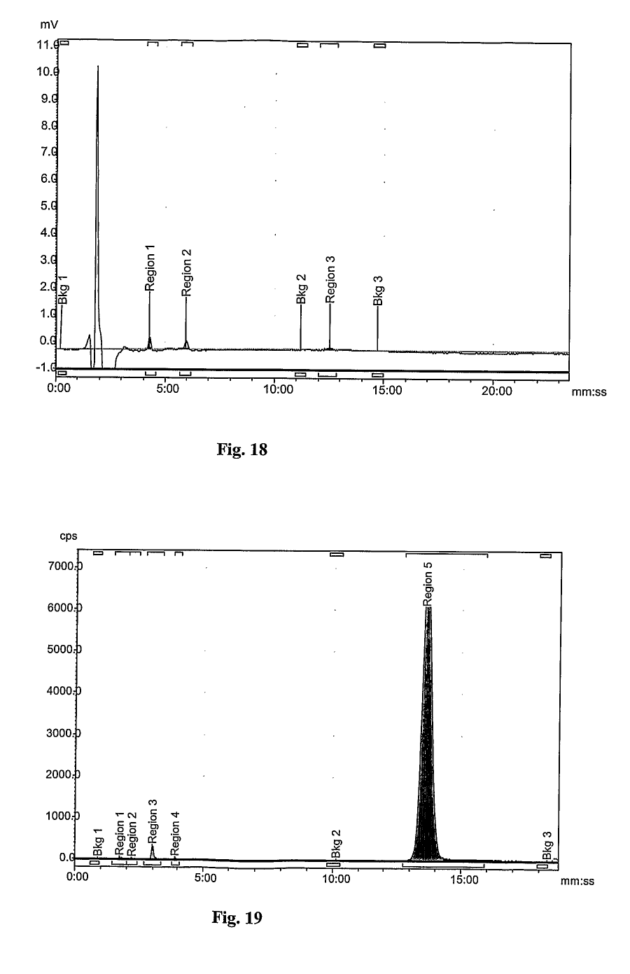

The UV chromatogram (226 nm) of the product is shown in FIG. 18 and data is shown in the table below.

| Retention | Height | Area | % Total | % ROI |
|---|---|---|---|---|
| (mm:ss) | (mV) | (mV) | (%) | (%) |
| 0:12 | −0.3 | | | |
| 4:15 | 0.2 | 3.5 | N/A | 43.38 |
| 5:58 | 0.0 | 2.8 | N/A | 34.63 |
| 11:11 | −0.2 | | | |
| 12:30 | −0.2 | 1.8 | N/A | 21.99 |
| 14:44 | −0.3 | | | |
| | | 8.1 | N/A | 100.00 |

The QC analysis revealed that both enantiomers in each of the formulations were stable with radiochemical purity ranging from 99.3% to 95.2% over a period of 4 h in saline.

Dulbecco's Phosphate Buffered Saline, pH 7: t=0

The F18 chromatogram of the product is shown in FIG. 19 and data is shown in the table below.

| Retention | Height | Area | % Total | % ROI |
|---|---|---|---|---|
| (mm:ss) | (cps) | (cps) | (%) | (%) |
| 0:52 | 8.0 | | | |
| 1:43 | 18.2 | 126.7 | 0.07 | 0.07 |
| 2:11 | 15.2 | 81.1 | 0.04 | 0.04 |
| 2:59 | 332.2 | 1681.6 | 0.87 | 0.88 |
| 3:53 | 23.6 | 84.8 | 0.04 | 0.04 |
| 10:09 | 8.5 | | | |
| 13:36 | 7124.1 | 189636.4 | 98.25 | 98.97 |
| 18:19 | 15.7 | | | |
| | | 191610.6 | 99.27 | 100.00 |

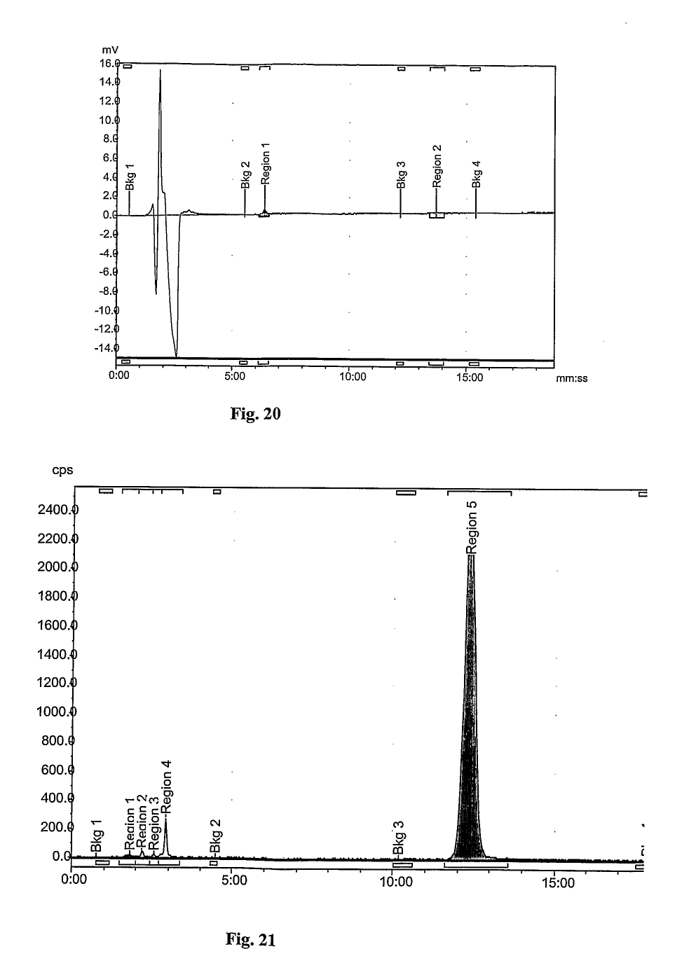

The UV chromatogram (226 nm) of the product is shown in FIG. 20 and data is shown in the table below.

| Retention | Height | Area | % Total | % ROI |
|---|---|---|---|---|
| (mm:ss) | (mV) | (mV) | (%) | (%) |
| 0:32 | 0.0 | | | |
| 5:32 | 0.3 | | | |
| 6:22 | 0.7 | 5.0 | N/A | 80.64 |
| 12:10 | 0.5 | | | |
| 13:43 | 0.5 | 1.2 | N/A | 19.36 |
| 15:25 | 0.5 | | | |
| | | 6.3 | N/A | 100.00 |

Dulbecco's Phosphate Buffered Saline, pH 7: t=4 h

The F18 chromatogram of the product is shown in FIG. 21 and data is shown in the table below.

| Retention | Height | Area | % Total | % ROI |
|---|---|---|---|---|
| (mm:ss) | (cps) | (cps) | (%) | (%) |
| 0:46 | 7.0 | | | |
| 1:49 | 24.3 | 235.1 | 0.39 | 0.40 |
| 2:10 | 46.6 | 302.0 | 0.51 | 0.51 |
| 2:32 | 27.4 | 144.1 | 0.24 | 0.24 |
| 2:54 | 282.1 | 1570.5 | 2.63 | 2.65 |
| 4:29 | 7.2 | | | |
| 10:10 | 8.5 | | | |
| 12:20 | 2468.6 | 56939.1 | 95.33 | 96.20 |
| 17:54 | 9.0 | | | |
| | | 59190.8 | 99.10 | 100.00 |

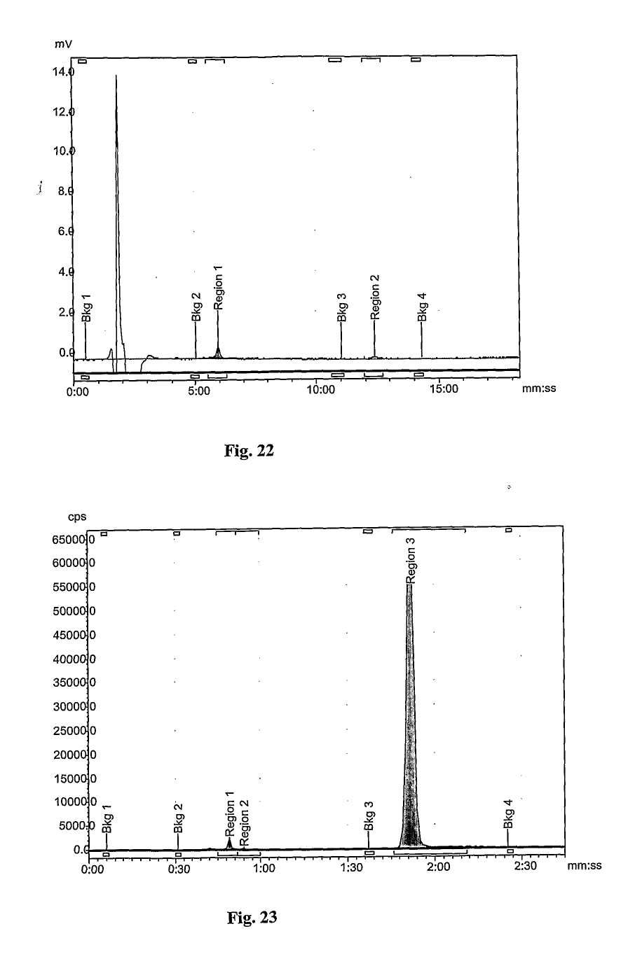

The UV chromatogram (226 nm) of the product is shown in FIG. 22 and data is shown in the table below.

| Retention | Height | Area | % Total | % ROI |
|---|---|---|---|---|
| (mm:ss) | (mV) | (mV) | (%) | (%) |
| 0:30 | −0.3 | | | |
| 5:02 | −0.3 | | | |
| 5:56 | 0.2 | 6.0 | N/A | 74.94 |
| 11:02 | −0.4 | | | |
| 12:23 | −0.3 | 2.0 | N/A | 25.06 |
| 14:18 | −0.4 | | | |
| | | 8.0 | N/A | 100.00 |

The QC analysis revealed that both enantiomers in each of the formulations were stable with radiochemical purity ranging from 99.0% to 96.2% over a period of 4 h in Dulbecco's phosphate buffered saline, pH 7.

PBS: t=0

The F18 chromatogram of the product is shown in FIG. 23 and data is shown in the table below.

| Retention | Height | Area | % Total | % ROI |
|---|---|---|---|---|
| (mm:ss) | (cps) | (cps) | (%) | (%) |
| 0:06 | 9.0 | | | |
| 0:31 | 7.0 | | | |
| 0:49 | 2097.8 | 2705.5 | 1.38 | 1.39 |
| 0:54 | 127.7 | 347.0 | 0.18 | 0.18 |
| 1:37 | 55.6 | | | |
| 1:52 | 64308.0 | 192215.5 | 97.95 | 98.44 |
| 2:25 | 78.2 | | | |
| | | 195267.9 | 99.51 | 100.00 |

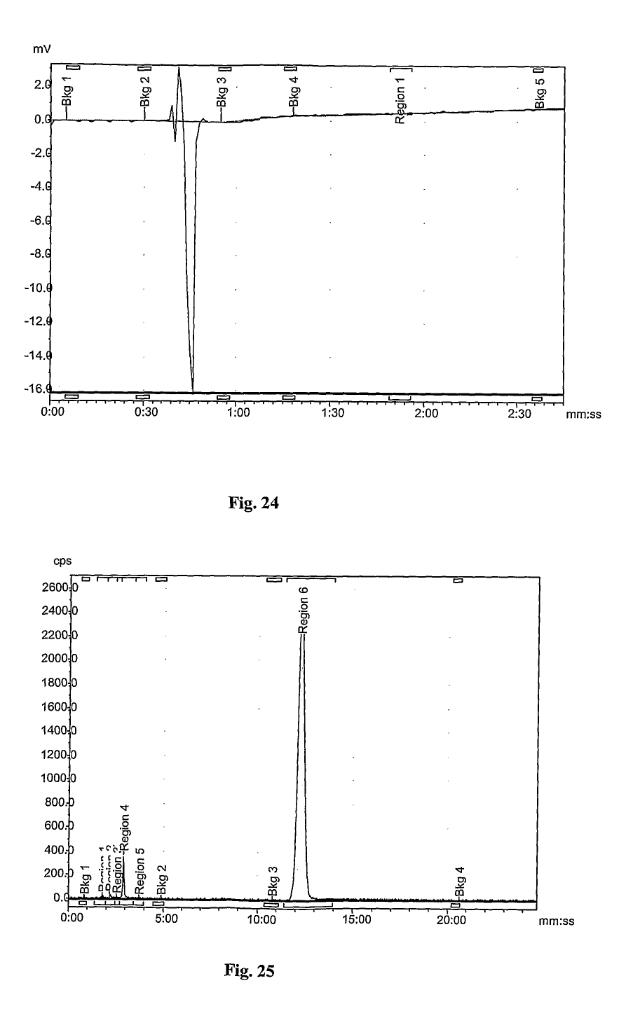

The UV chromatogram (226 nm) of the product is shown in FIG. 24 and data is shown in the table below.

| Retention | Height | Area | % Total | % ROI |
|---|---|---|---|---|
| (mm:ss) | (mV) | (mV) | (%) | (%) |
| 0:05 | 0.0 | | | |
| 0:30 | 0.0 | | | |
| 0:55 | −0.1 | | | |
| 1:18 | 0.3 | | | |
| 1:52 | 0.6 | 0.3 | N/A | 100.00 |
| 2:37 | 0.8 | | | |
| | | 0.3 | N/A | 100.00 |

PBS: t=4 h

The F18 chromatogram of the product is shown in FIG. 25 and data is shown in the table below.

| Retention | Height | Area | % Total | % ROI |
|---|---|---|---|---|
| (mm:ss) | (cps) | (cps) | (%) | (%) |
| 0:50 | 10.1 | | | |
| 1:46 | 25.3 | 179.0 | 0.28 | 0.29 |
| 2:10 | 40.5 | 195.4 | 0.31 | 0.31 |
| 2:31 | 20.3 | 88.2 | 0.14 | 0.14 |
| 2:53 | 369.7 | 1674.2 | 2.66 | 2.68 |
| 3:43 | 11.3 | 46.1 | 0.07 | 0.07 |
| 4:53 | 7.2 | | | |
| 10:47 | 9.6 | | | |
| 12:14 | 2599.9 | 60398.8 | 96.06 | 96.51 |
| 20:36 | 6.8 | | | |
| | | 62581.7 | 99.53 | 100.00 |

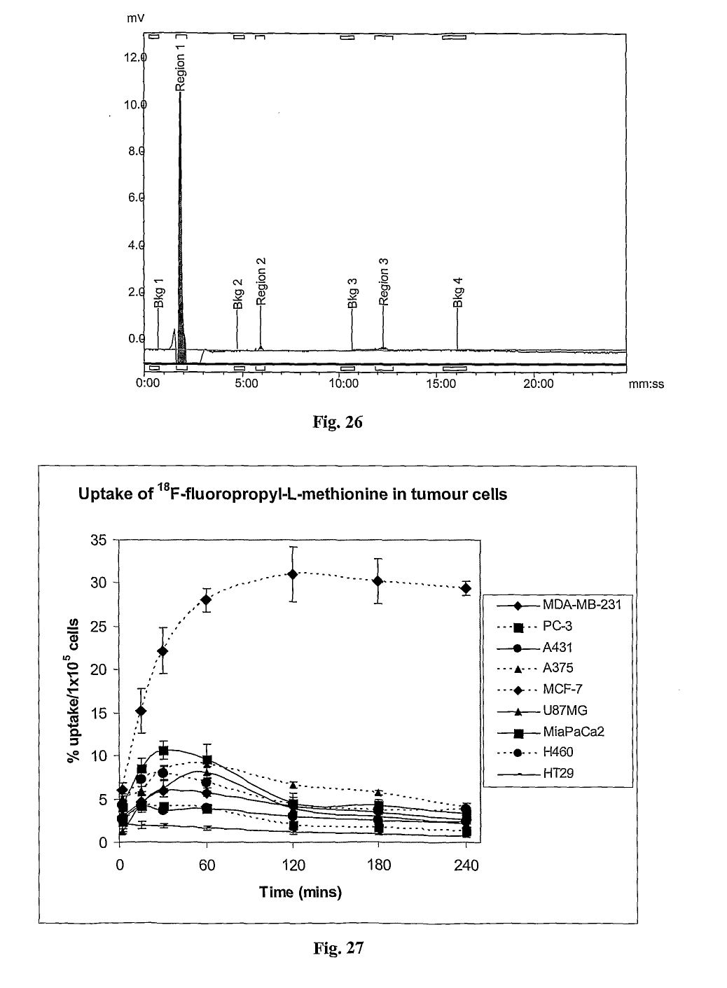

The UV chromatogram (226 nm) of the product is shown in FIG. 26 and data is shown in the table below.

| Retention | Height | Area | % Total | % ROI |
|---|---|---|---|---|
| (mm:ss) | (mV) | (mV) | (%) | (%) |
| 0:42 | −0.4 | | | |
| 1:48 | 13.1 | 161.8 | N/A | 97.36 |
| 4:43 | −0.4 | | | |
| 5:54 | −0.2 | 1.7 | N/A | 1.00 |
| 10:40 | −0.4 | | | |
| 12:18 | −0.3 | 2.7 | N/A | 1.65 |
| 16:03 | −0.4 | | | |
| | | 166.2 | N/A | 100.00 |

The QC analysis revealed that both enantiomers in each of the formulations were stable with radiochemical purity ranging from 98.4% to 96.5% over a period of 4 h in phosphate buffer.

### 3) Conclusions

[¹⁸F]-L-FPHCys and [¹⁸F]-D-FPHCys were synthesised in presence of potassium carbonate with a radiochemical purity>98% and an enantiomeric purity>98%. The QC analysis show that [¹⁸F]-L-FPHCys and [¹⁸F]-D-FPHCys were relatively stable in different aqueous solution over a period of 4 h with a radiochemical purity great of 95%.

### Activity Studies

### Methods:

### Cell Cultures

The human A375 (malignant melanoma), A431 (epidermoid carcinoma), HT29 (colorectal adenocarcinoma), MCF-7 (breast adenocarcinoma), MDA-MB-231 (breast adenocarcinoma), PC-3 (prostate adenocarcinoma) tumour cells were grown in RPMI-1640 medium supplemented with 10% fetal bovine serum (FBS) and 2 mM L-glutamine. The human H460 (large cell lung carcinoma) cell line was grown in RPMI-1640 supplemented with 10% FBS, 1 mM L-glutamine and 1 mM sodium pyruvate. The MiaPaCa2 human pancreatic carcinoma cell line was grown in Dulbecco's Modified Eagle medium supplemented with 10% FBS, 2.5% horse serum, 1 mM L-glutamine and 1 mM sodium pyruvate. The human U87MG glioblastoma cell line was cultured in Minimum Essential Medium supplemented with 10% FBS, 2 mM L-glutamine and 1 mM sodium pyruvate. Cells were maintained in 175 cm² flasks in a 5% CO₂, 37° C. humidified incubator. Cells were passaged routinely every 7 days and the media changed twice weekly.

One day prior to the experiments, cell cultures were trypsinised with a solution of 0.05% trypsin in PBS without Ca²⁺ and Mg²⁺ and containing 0.02% EDTA. Cells were seeded in 24-well culture dishes in complete medium and left to attach overnight. For MCF-7 cells, 3×10⁵ cells were seeded, for all other cell lines, 2.5×10⁵ cells were seeded.

### Cell Uptake Studies

On the day of the study, the cells were counted and the viability assessed in triplicate for each cell line. Before the incubation with radiolabelled FPM, cells were washed once with PBS containing Ca²⁺ and Mg²⁺ to remove all traces of the culture medium. The radiolabelled FPM was formulated in PBS containing Ca²⁺ and Mg²⁺. Aliquots of 500 μL (˜10 μCi) of freshly prepared FPM were added and the cells were incubated in triplicate at 37° C. for 2, 15, 30, 60, 120, 180 and 240 min. After stopping the tracer uptake with 1 mL of ice-cold PBS, cells were solubilised with 500 μL of 0.2N NaOH and the activity was measured with a gamma counter. The results were expressed as percent of applied dose per 1×10⁵ cells.

### Competitive Inhibition Studies

Inhibitor studies were carried out to investigate the transport mechanism involved in the uptake of FPM in tumour cells. The following amino acid transport inhibitors were used; 2-amino-bicyclo[2.2.1]-heptane-2-carboxylic acid (BCH) and N-methyl-α-aminoisobutyric acid (MeAIB) for the sodium-independent L type transport system and the A type amino acid transport system respectively.

On the day of the study, the cells were counted and the viability assessed in triplicate for each cell line. Before the incubation with radiolabelled FPM, cells were washed once with PBS containing Ca²⁺ and Mg²⁺ to remove all traces of the culture medium. Aliquots of 400 μL of inhibitor (12.5 mmol/L in PBS with Ca²⁺ and Mg²⁺) or PBS (for controls) were added to cells prior to the addition of radiolabelled FPM. Aliquots of 100 μL (about 10 μCi) of freshly prepared FPM were added and the cells were incubated in triplicate at 37° C. for 30 min. After stopping the tracer uptake with 1 mL of ice-cold PBS, cells were solubilised with 500 μL of 0.2N NaOH and the activity was measured with a gamma counter. The results were expressed as percent of applied dose per 1×10⁵ cells. The percentage of uptake in the treatment groups, relative to the control group was determined.

### Results:

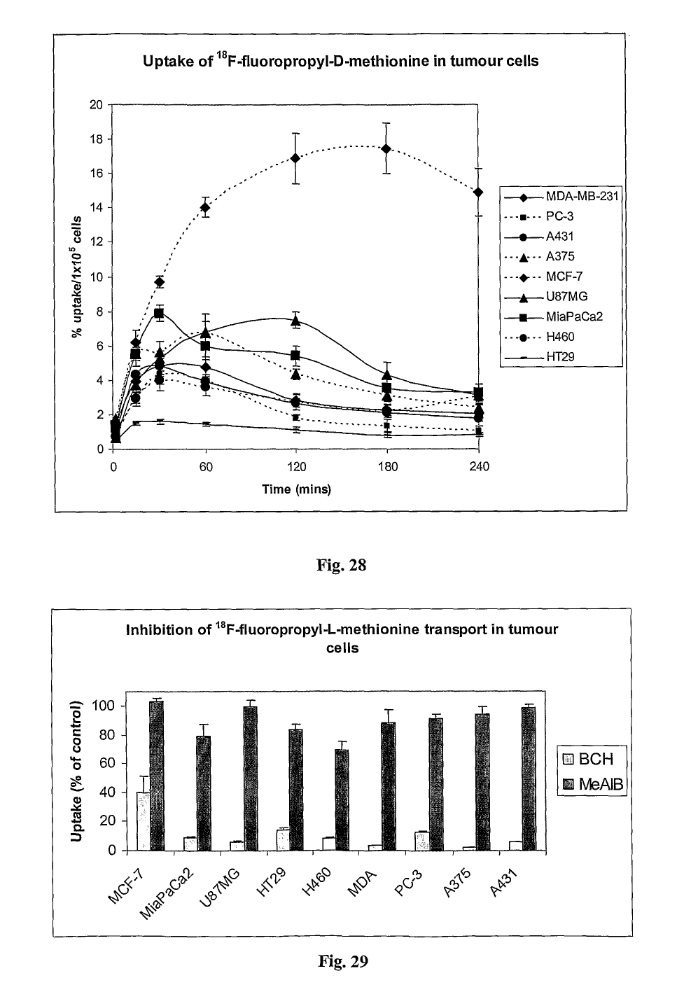

FIGS. 27 and 28 show uptake of ¹⁸F-fluoropropylmethionine (L and D respectively) in various types of tumour cells. Uptake of both D and L-FPM was highest in the MCF-7 breast tumour cell line. Uptake in this cell line increased for 120 min and then maintained uptake at the same level. Uptake in all other cell lines was significantly lower. Uptake in the pancreatic MiaPaCa2 tumour cell line peaked at 30 with both tracers before a decreasing. For both tracers, the HT29 colon cell line showed the lowest uptake. Uptake of L-FPM was higher in all cell lines compared to uptake of D-FPM.

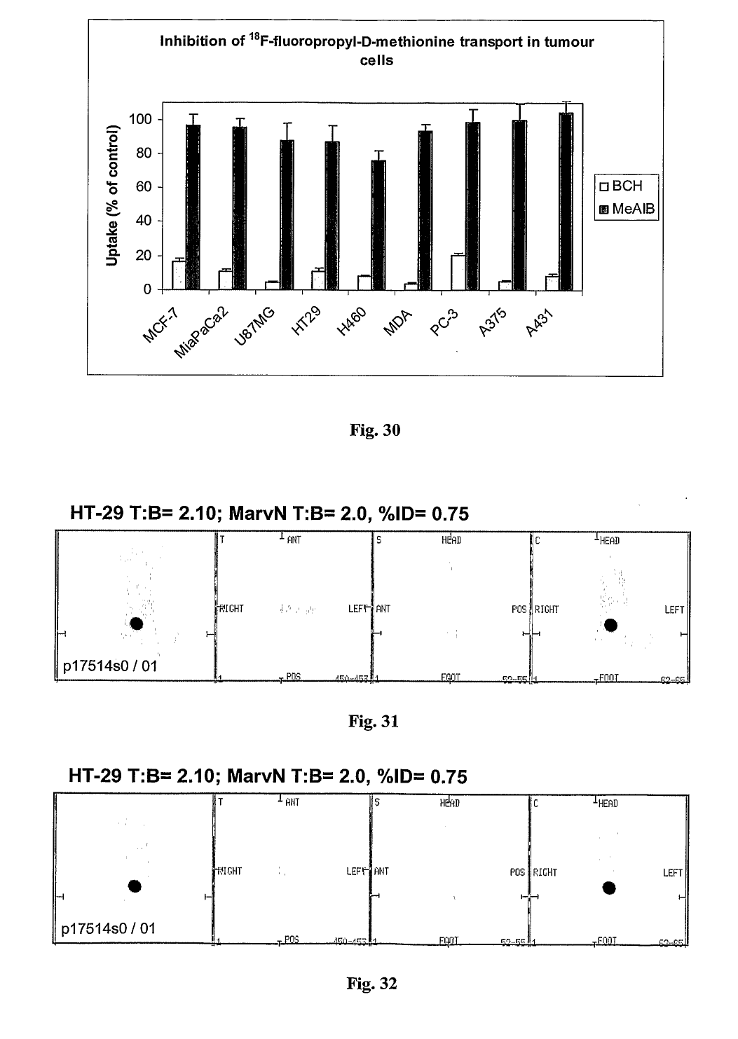

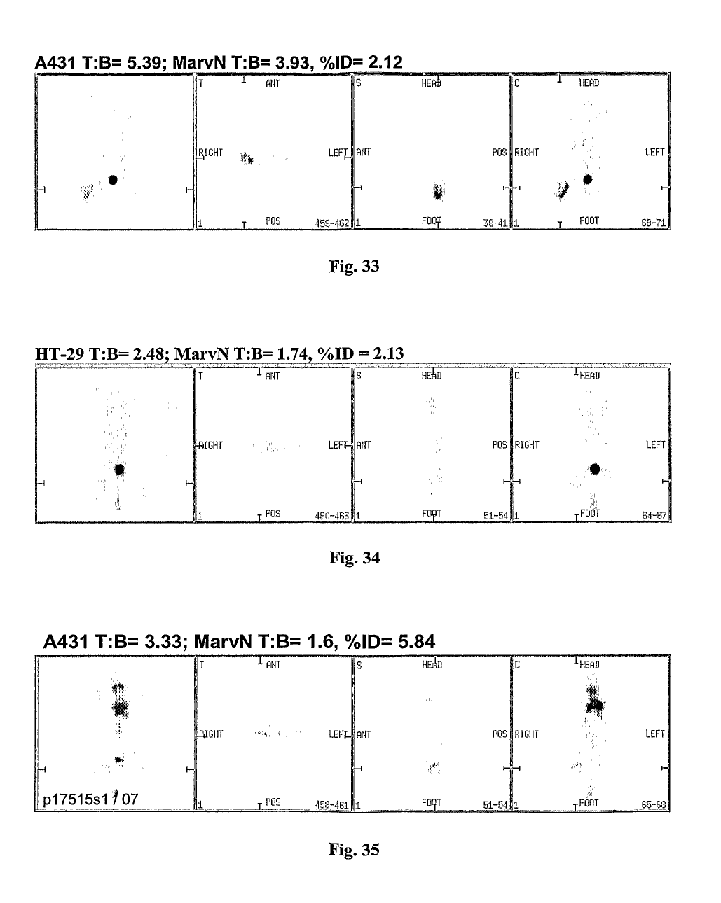

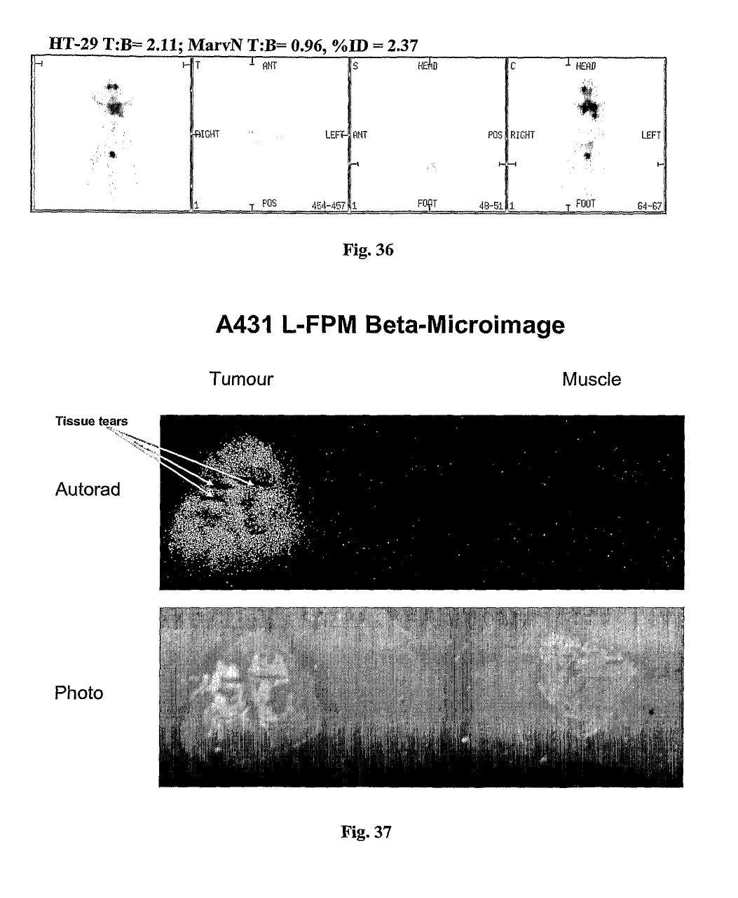

FIGS. 29 and 30 show inhibition of ¹⁸F-fluoropropylmethionine (L and D respectively) transport by known inhibitors. In all cell lines, uptake of both tracers was significantly inhibited in the presence of BCH. A mild reduction of L-FPM uptake was also observed in MiaPaCa2, HT29 and H460 cell lines in the presence of MeAIB (79%, 84% and 70% of control uptake respectively). Uptake of D-FPM in H460 cells was also reduced to 76% of control in the presence of MeAIB. These results indicate that D and L-FPM are predominantly taken up by cells via the sodium-independent L type transport system.

### In Vivo Imaging

In vivo imaging studies were performed on a Phillips Allegro™ small animal imaging system in mice implanted with A431 squamous cell carcinoma and HT-29 colon carcinoma xenografts on the left leg of Balb/c Nude mice. Approximately 400 μCi of each of D and L ¹⁸FPM in 100 μl saline were injected intravenously and PET imaging performed at 90 minutes post-tracer injection. A comparison with FDG (fluorodeoxyglucose) uptake was also performed.

### Conclusions

D- and L-FPM showed high uptake into tumour cells, particularly in the MCF-7 (breast), U87MG (glioma), MiaPaCa2 (colon) and A375 (melanoma) cell lines. Furthermore this high uptake in activity can be competitively inhibited by the amino acid transporter substrates BCH and MeAIB.

1.-30. (canceled)

31. A compound which is an ¹⁸F-radiolabelled S-propylhomocysteine or a derivative thereof, said compound having an enantiomeric purity of at least about 90%.

32. The compound of claim 31 which is ¹⁸F-radiolabelled on the S-propyl group.

33. The compound of claim 32 which is S-(3-[¹⁸F]fluoropropyl)homocysteine.

34. The compound of claim 32 which is an N-protected S-(3-[¹⁸F]fluoropropyl)homocysteine.

35. The compound of claim 32 which is C-protected as an ester.

36. The compound of claim 34 which is C-protected as an ester.

37. The compound of claim 31 which is the D-enantiomer.

38. The compound of claim 31 having a radiochemical purity of at least about 90%.

39. A process for making an ¹⁸F-radiolabelled S-propylhomocysteine comprising:
treating an N-protected ester of a substituted S-propylhomocysteine having a leaving group on the S-propyl group with a complexed ¹⁸F⁻ salt in the presence of a base to form a protected product, said ester having an enantiomeric purity of at least about 90% and said base being such that it does not cause racemisation of the protected product; and
deprotecting the protected product to form the ¹⁸F-radiolabelled S-propylhomocysteine.

40. The process of claim 39 wherein the N-protected ester is an N-t-butoxycarbonyl (Boc) protected ester.

41. The process of claim 39 wherein the N-protected ester is a t-butyl ester.

42. The process of claim 39 wherein the leaving group is in the 3 position of the S-propyl group.

43. The process of claim 39 wherein the N-protected ester of the substituted S-propylhomocysteine is that enantiomer which is capable of producing the D-enantiomer of the ¹⁸F-radiolabelled S-propylhomocysteine under the conditions of said process.

44. The process of claim 39 wherein the leaving group is chloride, bromide or tosylate.

45. The process of claim 39 wherein the base is an oxalate salt.

46. The process of claim 45 wherein the base is potassium oxalate.

47. The process of claim 39 wherein the step of deprotecting comprises treating the protected product with a strong acid.

48. The process of claim 39 wherein the complexed ¹⁸F⁻ salt is a cryptand-complexed ¹⁸F⁻ salt.

49. The process of claim 48 wherein the cryptand-complexed ¹⁸F⁻ salt is a 4,7,13,16,21,24-hexaoxa-1,10-diazabicyclo[8.8.8]-hexacosane-complexed ¹⁸F⁻ salt.

50. The process of claim 39 wherein the step of deprotecting is conducted without separation of the protected product.

51. The process of claim 39 comprising the step of making the N-protected ester of the substituted S-propylhomocysteine having a leaving group on the S-propyl group, said step comprising reacting an N-protected homocysteine ester with a 1-bromopropane having a substituent thereon.

52. The process of claim 51 wherein either:
the substituent on the 1-bromopropane is the leaving group, or
the substituent on the 1-bromopropane is an OH group and the step of making the N-protected ester of the substituted S-propylhomocysteine having a leaving group on the S-propyl group additionally comprises tosylating the OH group.

53. The process of claim 51 comprising making the N-protected homocysteine ester from homocysteine.

54. The process of claim 39 additionally comprising purifying the ¹⁸F-radiolabelled S-propylhomocysteine.

55. The process of claim 39 which produces the ¹⁸F-radiolabelled S-propylhomocysteine in an enantiomeric purity of at least about 90%.

56. The process of claim 39 which produces the ¹⁸F-radiolabelled S-propylhomocysteine in a radiochemical purity of at least about 90%.

57. The process of claim 39 which is conducted using an automatic synthesiser.

58. Use of the compound of claim 31 as a radiotracer in tumour diagnosis.

59. Use of the compound of claim 31 in the manufacture of a composition for use in positron emission tomography.

60. A composition for use in positron emission tomography, said composition comprising the compound of claim 31 and a clinically acceptable carrier.

61. The composition of claim 60 wherein the carrier is aqueous.
